(19) United States
(12) Patent Application Publication
(10) Pub. No.: US 2018/0051301 A1
(43) Pub. Date: Feb. 22, 2018

(54) INDUCTION OF PACEMAKER-LIKE CELLS FROM CARDIOMYOCYTES

(71) Applicant: WASHINGTON UNIVERSITY, St. Louis, MO (US)

(72) Inventors: Stacey Rentschler, St. Louis, MO (US); Igor Efimov, St. Louis, MO (US); David Curiel, St. Louis, MO (US)

(21) Appl. No.: 15/554,371

(22) PCT Filed: Mar. 2, 2016
(86) PCT No.: PCT/US2016/020473

§ 371 Date: Aug. 29, 2017

Related U.S. Application Data
(60) Provisional application No. 62/127,010, filed on Mar. 2, 2015.

(51) Int. Cl. C12N 15/867

(52) U.S. Cl. CPC C12N 15/867; A61K 48/00

(57) ABSTRACT

Disclosed are compositions and methods relating to the cardiac conduction system. Compositions, methods for converting cardiac tissue to an induced-sinoatrial node, and methods for treating sinus node dysfunction (SND) in an individual in need thereof are disclosed. Also disclosed are compositions and methods for evaluating virally-transduced cardiac tissue.

### CROSS-REFERENCE TO RELATED APPLICATIONS

This application claims the benefit to U.S. Provisional Application Ser. No. 62/127,010, filed Mar. 2, 2015, which is incorporated herein by reference in its entirety.

### STATEMENT IN SUPPORT FOR FILING A SEQUENCE LISTING

A computer readable form of the Sequence Listing containing the file named “WUSTL015314PCT_ST25.txt”, which is 95,430 bytes in size (as measured in MICROSOFT WINDOWS® EXPLORER), is provided herein and is herein incorporated by reference. This Sequence Listing consists of SEQ ID NOs:1-7.

### BACKGROUND OF THE DISCLOSURE

The present disclosure relates generally to treating diseases of the cardiac conduction system. More particularly, the present disclosure relates to compositions and methods for converting cardiac tissue into pacemaker-like cells, and methods for treating sinus node dysfunction. The present disclosure further relates to compositions and methods for evaluating candidate cardiac therapies.

The heartbeat originates in the sinoatrial node (SAN), a small structure located in the right atrium. The SAN contains specialized cardiomyocytes, the pacemaker cells, which spontaneously depolarize via mechanisms regulated by distinct ion channel (voltage clock) and calcium handling protein (Ca²⁺ clock) expression within the node. The currently held paradigm is that cardiac automaticity arises from the integrated activity of voltage-gated ionic currents (I_{f}, I_{Ca,L} and I_{Ca,T}) and intracellular Ca²⁺ cycling regulated by transporters (Na⁺-Ca²⁺ exchanger, NCX) and sarcoplasmic reticulum Ca²⁺ release. The SAN has a unique anatomical structure, including a fibrous insulation surrounding the nodal cells, decreased gap junction coupling of nodal cells with neighboring atrial cardiomyocytes, and specialized conduction pathways. This anatomic and molecular architecture of the SAN ensures electrical source-sink matching, preventing the node from becoming hyperpolarized and quiescent by the surrounding atrial cardiomyocytes, as well as allowing conduction of action potentials from the SAN to nearby atrial myocardium.

Sinus node dysfunction (SND) is a common cardiac conduction abnormality that can result in decreased heart rate, fatigue, syncope, and even sudden death. It is estimated to affect at least 1 out of every 600 adults over age 65. While less common, sinus node dysfunction is also prevalent in the pediatric population where it can result from developmental defects or, more commonly, from surgical trauma during the repair of congenital heart defects. While overall reliable and effective, implanted electronic pacemakers are subject to several limitations, including suboptimal autonomic responsiveness, fixed size and length, susceptibility to infection, mechanical failure, and a finite battery life. In the pediatric population, some of these limitations are particularly troubling. For example, current device longevity is on the order of 10 years, which means that a pediatric patient implanted at the age of 10 can expect to have at least 7 generator changes during their lifespan. While the acute procedural risk associated with each generator change may be minimal, the risk of infection increases with each subsequent procedure. In addition, while electronic pacemakers provide some degree of rate-responsiveness, the technology is predominantly accelerometer based and not sensitive to autonomic input, which means that heart rate will not increase with activities such as isometric exercise or emotional stressors. Inadequate matching of heart rate with exercise intensity can prevent children with electronic devices from participating in high-intensity sports. An additional consideration relates to placement of transvenous leads, which are more prone to fracture and failure in the active pediatric population. Given that leads are appropriately sized to the patient at the time of implant, they often fail due to dislodgement or mechanical stress, which may require lead extraction and can place children at risk of venous stenosis. A final consideration especially poignant in the pediatric patient are those who are unable to undergo transvenous access due to intracardiac shunts, mechanical valve prostheses, or other cardiac anatomical defects that preclude transvenous access, such as after a Fontan procedure. This often results in placement of an epicardial pacing system and its attendant limitations, including decreased lead longevity and abdominal pulse generator location.

Other therapies for cardiac pacemaking rely on drug therapy and ablation. Antiarrhythmic drugs are widely prescribed and used, but may result in adverse systemic side effects. Ablation involves permanent removal of the tissue identified as the source of, or critical to, the maintenance of the arrhythmias. Ablation has exhibited success in the treatment of atrioventricular node reentry tachycardia, accessory pathway tachycardia, and atrial flutter, but is less successful in the treatment of other arrhythmias such as atrial fibrillation (AF) and ventricular tachycardia (VT), and is not useful in the treatment of bradycardia.

Cell-based approaches have presented several challenges thus far, including difficulties with engraftment of sufficient numbers of differentiated cardiomyocytes within existing myocardium. Viral-based delivery systems have been used to express the molecular determinants of the pacemaker current. While there has been some degree of success in approximating physiologic pacemakers using this approach, a major limitation is the need for stable and long-lasting expression of exogenous gene products to maintain the active pacemaker.

To date, over 1800 gene therapy clinical trials have been completed, are ongoing or have been approved worldwide, indicating a wide-ranging acceptance of gene therapy-based treatments (Ginn et al. 2013. J. Gene Med. 15:65-77). The rapidly changing field of gene therapy promises a number of innovative treatments for patients. Owing to the rapid and mature development of emerging biotechnology in the fields of cell culture, cell preservation, and recombinant DNA technology, more and more gene medicinal therapy products have been approved for marketing to treat serious diseases that have been challenging to treat with current medical practice or medicine. Past and current scientific advances have facilitated development of selectively targeted vectors that are efficient in gene transfer and reduce off-target effects. The field has also reacted positively to the occurrence of vector-related adverse events and other lessons learned from earlier gene therapy trials, including the challenges posed by the immune system. These lessons are now being accommodated in an iterative manner in ongoing trial activity.

Substantial barriers exist that impede the translation of breakthroughs tested in animal models into humans, including scarcity of in vitro human model systems for evaluation of putative therapies. Organotypic culture is a widely used technique to study diverse organ systems, including the brain, kidney and liver.

Adenovirus is non-integrating and efficiently produces high-titer virus. Replication-deficient adenoviruses have previously been tested on several mouse and human tissues and are currently being evaluated in several clinical trials.

These shortcomings highlight the need to develop biological pacemakers by engineering tissue to endow pacemaker properties to regions of the heart using viral-based gene delivery or exogenous cell-based approaches. Accordingly, there exists a need to develop compositions and methods for cardiac pacemaking therapies, as well as compositions and methods for assessing candidate cardiac pacemaking therapies.

### BRIEF DESCRIPTION OF THE DISCLOSURE

The present disclosure relates generally to treating diseases of the cardiac conduction system. More particularly, the present disclosure relates to compositions and methods for converting cardiac tissue into pacemaker-like cells, and methods for treating sinus node dysfunction. The present disclosure further relates to compositions and methods for evaluating candidate cardiac therapies.

In one aspect, the present disclosure is directed to a composition comprising: an adenovirus encoding a transcription factor, wherein the transcription factor comprises a β-catenin, T-box18 (Tbx18), short stature homeobox 2 (Shox2), Islet-1, and combinations thereof.

In another aspect, the present disclosure is directed to a method for converting a cardiomyocyte into an induced-sinoatrial node (iSAN) cell, the method comprising: infecting a cardiomyocyte with a composition comprising an adenovirus encoding a transcription factor, wherein the transcription factor comprises a β-catenin, T-box18 (Tbx18), short stature homeobox 2 (Shox2), Islet-1 (ISL1), and combinations thereof.

In another aspect, the present disclosure is directed to method for treating sinus node dysfunction (SND) in an individual in need thereof, the method comprising: administering to the individual a composition comprising an adenovirus encoding a transcription factor, wherein the transcription factor comprises a β-catenin, T-box18 (Tbx18), short stature homeobox 2 (Shox2), Islet-1 (ISL1), and combinations thereof.

In another aspect, the present disclosure is directed to a method for treating sinus node dysfunction (SND) in an individual in need thereof, the method comprising: administering to the individual a composition comprising an adenovirus encoding a transcription factor, wherein the transcription factor comprises a β-catenin, T-box18 (Tbx18), short stature homeobox 2 (Shox2), Islet-1 (ISL1), and combinations thereof.

In another aspect, the present disclosure is directed to an adenovirus encoding a transcription factor, wherein the adenovirus comprises a modified trimeric fiber protein and encodes a cardiac tissue-specific promoter.

In another aspect, the present disclosure is directed to a method for converting cardiac tissue to an induced-sinoatrial node, the method comprising contacting the cardiac tissue with a composition comprising an adenovirus encoding a transcription factor, wherein the transcription factor comprises a β-catenin, T-box18 (Tbx18), short stature homeobox 2 (Shox2), Islet-1 (ISL1), and combinations thereof.

In another aspect, the present disclosure is directed to a cardiac slice culture system comprising: a cardiac tissue slice; and an incubation apparatus.

In another aspect, the present disclosure is directed to a method for evaluating candidate cardiac therapies. The method comprises: providing a cardiac tissue slice; contacting the cardiac tissue slice with a candidate cardiac therapy; and analyzing the cardiac tissue slice.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A and 1B depict the expression of the canonical Wnt reporter Axin2^{LacZ} within the newborn SAN.

FIGS. 1C-1D depict representative left ventricular electrical activation patterns during epicardial stimulation in littermate control (FIG. 1C) and Wnt GOF mice (FIG. 1D) demonstrating that ectopic Wnt activation programs ventricular myocytes to adopt a nodal-like electrical phenotype. Note different time scales in FIGS. 1C and 1D.

FIG. 1E is a graph depicting slower longitudinal conduction velocity in Wnt gain-of-function (GOF) mice as compared to littermate controls during stimulation at each cycle length. The difference between the two genotypes becomes greater at faster pacing rates (111 and 100 ms cycle lengths, n=4), which is consistent with decremental conduction, a property of nodal tissue. *p<0.05.

FIG. 2A depicts gene expression analysis in left atrial tissue of littermate control and Wnt GOF mice by qRT-PCR demonstrating robust activation of Axin2, a direct target of canonical Wnt signaling, and down-regulation of Scn5a encoding Nav1.5 in Wnt GOF mice. p=0.000004 in Axin2; p=0.0002 in Scn5a; p=0.18 in Tbx3.

FIG. 2B depicts transient Wnt activation in atrial myocardium significantly slowed atrial conduction, as evidenced by prolongation of p wave duration. p=0.0002.

FIG. 3A depicts a timeline of the human heart organotypic slice model protocol.

FIG. 3B depicts representative optical action potential (AP) curves from control untreated ventricular slices, slices treated acutely with the α₁-AR agonist phenylephrine (15 minutes), control cultured slices (24 hours), acute treatment with α₁-AR agonist after one day in culture (15 minutes) and chronic α₁-AR agonist treatment (24 hours) demonstrating acute and chronic therapeutic effects in the human heart organotypic slice model.

FIGS. 3C and 3D depict restitution curves for control, acute treated, and chronic treated ventricular slices demonstrating that acute α₁-AR stimulation at baseline as well as treatment after one day in culture shifted the AP restitution curve upwards significantly, while chronic α₁-AR stimulation shifted the AP restitution curve downwards. No effect of culture on restitution properties was noted.

FIG. 3E depicts the calculation of statistical difference for AP duration between each condition at 1 Hz (60 BPM) pacing frequency, demonstrating similar AP duration (APD) lengthening with acute phenylephrine treatment at baseline, as well as after slices were cultured for one day. Chronic phenylephrine treatment resulted in APD shortening. No effect of culture on APD was noted.

FIG. 3F depicts transverse conduction velocity for each condition demonstrating that acute α₁-AR stimulation at baseline and after culture for one day decreased CV, while chronic α₁-AR stimulation increased CV. There was no effect of culture on CV.

FIG. 4A depicts an intact human atrial preparation prior to sectioning for human atrial organotypic culture.

FIG. 4B depicts representative CaT recorded from various regions of a human atrial slice.

FIG. 4C depicts CaT recorded from distinct atrial regions demonstrating heterogeneity in the CaT morphology.

FIG. 4D depicts atrial slice pacing containing SAN cells at various frequencies (0.5 Hz/30 BPM upper traces, 1 Hz/60 BPM middle, and no pacing lower traces) and recording membrane voltage (Vm) demonstrates normal sinus node physiology, with both spontaneous (automaticity) and captured APs (stimulation).

FIGS. 5A-5C are fluorescence images depicting prolonged adenovirus mediated gene expression in adult human ventricular slices transduced with adenovirus encoding eGFP after tissue harvest at Day 0 (left panel) and Day 4 (right panel). Top panels in FIG. 5A depict boxed regions in FIG. 5B at higher magnification showing the green channel for eGFP (left images in SA at Day 0 and Day 4) and the red channel (right images in 5a at Day 0 and Day 4) for α-actinin staining (to visualize sarcomeric structure in cardiomyocytes). FIG. 5B are merged images depicting eGFP, α-actinin staining and DAPI (to visualize nuclei). FIG. 5C are images depicting immunohistochemical staining for connexin 43 (Cx 43) demonstrating maintenance of gap junctions at the intercalated disc throughout prolonged slice culture (indicated by white arrowheads).

FIG. 6 is a schematic illustrating adenovirus Ad5 modification to redirect the Ad vector to an alternative cell surface receptor (αᵥ integrins) present on adult human cardiomyocytes by genetically modifying the fiber knob domain to express the RGD-4C peptide.

FIG. 7A are whole mount images depicting the surface of ventricular slices treated with 1.1 E10 viral particles of Ad5 (upper panel) or Ad5-RGD (lower panel) encoding EGFP after 48 hours in culture.

FIG. 7B are histologic sections through the slices (Ad5, upper panel or Ad5-RGD, lower panel) demonstrating virally-transduced EGFP⁺ cells near the culture surface with co-staining for α-actinin to delineate cardiomyocytes.

FIGS. 7C and 7D are regions from FIG. 7B shown at higher magnification depicting α-actinin staining (FIG. 7C), and α-actinin, eGFP, and DAPI co-staining (FIG. 7D) demonstrating Ad5 (upper panel) transduced a low number of α-actinin⁺ cardiomyocytes, as well as a significant number of non-cardiomyoyctes (white arrowheads), while Ad5-RGD (lower panel) greatly enhanced the number of eGFP cardiomyocytes when applied at the same viral titer.

FIG. 7E are higher magnification images of the boxed regions in FIG. 7D depicting eGFP (upper panel) and α-actinin (lower panel) within cardiomyocytes, which had a well-preserved sarcomeric structure throughout prolonged culture. Images are representative of n=4 donor hearts with n=3 slices virally transduced per heart.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The approach of the present disclosure is to directly convert endogenous cardiomyocytes in situ into induced-SAN (iSAN) cells. An important advantage of this approach is that once cellular reprogramming has occurred, continuous expression of the reprogramming genes is not required for maintenance of the pacemaker. In addition, regulation of the entire family of currents that control impulse initiation can be accomplished.

As used herein, “atrium” and “atrial tissue” are used according to their ordinary meanings known to those skilled in the art (such as medical and research professionals) to refer to and include Sinus node, crista terminalis, free walls, appendages, atrioventricular junction, and atrioventricular node. Similarly, “ventricle” and “ventricular tissue” are used according to their ordinary meanings known to those skilled in the art (such as medical and research professionals) to refer to and include atrioventricular junction, His bundle, bundle branches, Purkinje cells, free walls, septum, and outflow tract.

As used herein, “individual in need thereof” refers to an individual susceptible to or at risk of a specified disease, disorder, or condition. More particularly, in the present disclosure the methods of converting cardiomyocytes into pacemaker-like cells, such as induced-sinoatrial node (iSAN) cells, can be used with an individual or subset of individuals who have, are susceptible to, and at elevated risk for experiencing diseases of the cardiac conduction system (e.g., sinus node dysfunction), an individual or subset of individuals who are susceptible to or at elevated risk for having cardiac tissue exhibiting abnormal electrical activity, an individual or subset of individuals who are susceptible to or at elevated risk for sick sinus syndrome, an individual or subset of individuals who are susceptible to or at elevated risk for sinus bradycardia, an individual or subset of individuals who are susceptible to or at elevated risk for tachycardia-bradycardia syndrome, an individual or subset of individuals who are susceptible to or at elevated risk for atrial fibrillation, an individual or subset of individuals who are susceptible to or at elevated risk for atrioventricular block, an individual or subset of individuals who are susceptible to or at elevated risk for chronotropic incompetence, an individual or subset of individuals who are susceptible to or at elevated risk for prolonged QT syndrome, and an individual or subset of individuals who are susceptible to or at elevated risk for heart failure. In some embodiments, the compositions and methods disclosed herein are used to treat individuals having or susceptible to a disease or disorder such as a cardiac-related syncope (e.g., Stokes-Adam syncope), an abnormality of sinus node function such as persistent sinus bradycardia, sino-atrial (S-A) block manifested as S-A Wenckebach, complete S-A block or sinus arrest, and high-grade atrioventricular block; or bradycardia-tachycardia syndrome or other bradycardia related condition. Particularly suitable individuals in need thereof include those in need of a biological pacemaker function. Suitable individuals in need thereof include those having an electronic pacemaker to modify the electrical activity of the individual's cardiac tissue. In some embodiments, the generation of the biological pacemaker supplements the function of the electronic pacemaker. In some embodiments, the generation of the biological pacemaker replaces the function of the electronic pacemaker such that the electronic pacemaker can be eliminated. In some embodiments, an electronic pacemaker is used to provide a short-term bridge to allow the generated biological pacemaker to take full functional effect in an individual. In some embodiments, a pharmacological agent that is used to treat cardiac arrhythmia is administered with the one or more transcription factors to provide a short-term bridge to allow the generated biological pacemaker to take full functional effect in an individual. Individuals may be susceptible to or at elevated risk for these diseases, disorders or conditions due to family history, age, environment, and/or lifestyle.

The individual in need thereof can be an adult individual, a child, and a pediatric individual. Particularly suitable individuals can be humans. Other particularly suitable individuals can be experimental animals such as rodents (e.g., mice and rats), pigs, primates, rabbits, cows, horses, dogs, and the like.

As used herein, the term “converting” refers to reprogramming cardiomyocytes to become biological pacemaker-like cells. Converting a cell to a natural pacemaker-like state allows for the converted cells to operate at naturally defined frequencies. As a result, the methods of the present disclosure require fewer administrations (or doses) of the compositions to achieve conversion of a sufficient number of cells to generate a new pacemaker in the heart and cardiac tissue. Further, the compositions and methods of the instant application can “reprogram” the cardiac tissue which allows for the maintenance of pacemaker-like activity even after the genes encoding the transcription factors themselves are no longer expressed.

As used herein, the terms “fragment” and “functional fragment” refer to a portion of an amino acid sequence (or polynucleotide encoding that sequence) having about 70%, preferably about 80%, more preferably about 90%, about 95%, about 96%, about 97%, about 98% or about 99% of the function of the corresponding full-length amino acid sequence (or polynucleotide encoding that sequence). Methods of detecting and quantifying functionality of such fragments and functional fragments include, for example, the heart slice method described herein.

The compositions and methods disclosed herein modulate cardiac rhythm. For example, the compositions and methods disclosed herein achieve a heart rate within about 25%, within about 20%, within about 15%, within about 10%, within about 5%, within about 2%, or within about 1% of a clinically desired heart rate as determined by those skilled in the art such as a medical professional.

In one aspect, the present disclosure is directed to a composition including: an adenovirus encoding a first transcription factor wherein the transcription factor includes a β-catenin, T-box18 (Tbx18), short stature homeobox 2 (Shox2), Islet-1 (ISL1), and combinations thereof.

In one embodiment, the composition includes an adenovirus encoding a first transcription factor wherein the transcription factor is a β-catenin; and at least a second transcription factor.

Particularly suitable transcription factors can be a β-catenin, T-box 18 (Tbx18), short stature homeobox 2 (Shox2), Islet-1 (ISL1, ISL LIM Homeobox 1), and combinations thereof. Particularly suitable combinations include, for example, β-catenin+Tbx18, β-catenin+Shox2, β-catenin+ISL1, β-catenin+Tbx18+Shox2, β-catenin+Tbx18+Shox2+ISL1, β-catenin+Tbx18+ISL1, and β-catenin+Shox2+ISL1. Other suitable transcription factors include, for example, T-box 3 (Tbx3) and canonical Wnt. As used herein, combinations of transcription factors can occur in any order when a combination of transcription factors is encoded by a single vector. For example, a combination of β-catenin+Tbx18 can be oriented in the vector as β-catenin-Tbx18 or Tbx18-β-catenin. In additional embodiments, related family members of these transcription factors can be used. In some embodiments, human transcription factors are particularly suitable. In other embodiments, homologs from different species are used (either in place of or in conjunction with the human transcription factor(s)). In some embodiments, the transcription factors are administered separately. In some embodiments, the transcription factors are administered at discrete time frames. In some embodiments, the transcription factors are administered in overlapping time frames. In some embodiments, a functional fragment of one transcription factor is used in conjunction with one (or more) full length transcription factors. In other embodiments, combinations of functional fragments of transcription factors are used.

Particularly suitable β-catenins can be constitutively active β-catenins. Constitutively active β-catenins include β-catenins having a S33A substitution, a S37A substitution, a T41A substitution, a S45A substitution, and combinations thereof (see, H. sapiens beta catenin gene GI:38519; Accession Z19054; Accession Version Z19054.1). A particularly suitable constitutively active β-catenin includes a S33A substitution, a S37A substitution, a T41A substitution, and a S45A substitution. These substitutions prevent phosphorylation of the specified amino residue, which results in the constitutively active β-catenin.

The one or more transcription factors induce an increase in the spontaneous, repetitive electrical activity of the cells, wherein the increase in the spontaneous, repetitive electrical activity of the cells is capable of generating an ectopic contraction of the cells.

Particularly suitable adenovirus can be a polycistronic adenovirus. Another particularly suitable adenovirus can be a replication-defective adenovirus.

A particularly suitable adenovirus is Ad serotype 5 (Ad5). Ad5 cellular entry is mediated by distinct binding and internalization events: the knob domain of trimeric fiber protein initiates attachment through interactions with coxsackie virus and Ad receptor (CAR), while internalization is mediated by interactions between αᵥβ3 and αᵥβ5 integrins and the arginine-glycine-aspartic acid (RGD) motif within the penton protein loop. Ad5 vectors of the present disclosure include a genetic targeting platform based on fiber knob modifications to confer Ad5 vectors a CAR-independent tropism.

Particularly suitable adenovirus modifications include “gutless adenovirus” which enable efficient immune-system evasion and avoid toxicity, thereby allowing for the durability of an induced-pacemaker. Gutless adenovirus or gutted Ad are devoid of all coding viral regions and can accommodate up to 36 kb of DNA. The gutless adenovirus only keeps the 5′ and 3′ inverted terminal repeats (ITRs) and the packaging signal (Psi) from the wild-type adenovirus. Because of the deletion of all viral coding genes, the gutless Ad vectors of the present disclosure are not targeted by anti-adenoviral immune response. This facilitates long-term transgene expression in the cardiac tissue. The gutless Ad vectors of the present disclosure are advantageous because of their ability to encode large and complex expression cassette (e.g., the inducible TetOn system). Use of tetracycline- (doxycycline-) inducible, polycistronic, gutless vectors can address stable conversion to an iSAN in the absence of ongoing transcription factor expression. As an additional benefit of this strategy, expression of transcription factors only in the presence of a well-tolerated drug (such as tetracycline and doxycycline) together with the use of tissue-specific promoters avoid off-target effects. Use of the TetOn and TetOff inducible systems advantageously allows for tight regulation over transcription factor expression. Further combination with a cardiac-tissue specific promoter provides the added advantage of transcription factor expression in the desired cardiac tissue. In addition to these advantages, the Ad virus of the present disclosure advantageously enhance the safety of the therapeutic strategy of the present disclosure.

The adenovirus can further include a modification of the viral capsid. Particularly suitable modifications can be an RGD sequence; polylysine residues; a serotype Ad5 knob and a serotype Ad3 knob; a binding motif that specifically binds to a cell-surface glycan; an antibody that specifically binds to a cardiomyocyte cell-surface receptor; a CD47 peptide; and combinations thereof. A particularly suitable CD47 peptide can be a humanized CD47 peptide.

Suitable fiber knob modifications include, for example, addition of a cysteine-constrained RGD-4C (CDCRGDCFC; SEQ ID NO:1) peptide that binds αᵥ integrins expressed on human cardiomyocytes with high affinity; addition of polylysine residues that adhere to cell-surface proteins containing polyanion motifs such as heparin sulfate receptors; Ad5/3 vector containing chimeric fibers composed of the tail and shaft domains of Ad5 with the knob domain of Ad serotype 3; and chimeric fiber modifications enabling targeting of specific cell-surface glycans. These genetic modifications of fiber knob domain provide enhanced virus infectivity in CAR-deficient tissues.

In other embodiments, an Ad capsid modification strategy based on fiber knob replacement with heterologous trimerization domain allowing incorporation of targeting ligand able to recognize a specific cardiomyocyte receptor can be used. A particularly suitable Ad capsid modification includes, for example, modifications to direct Ad binding to phagocyte receptor CD172α (also known as signal-regulatory protein alpha, SIRPα), a marker expressed specifically on cardiomyocytes derived from human embryonic stem cells. To modify the Ad capsid, the Ad knob domain can be replaced with a 95 amino acid trimerization domain of the T4 phage fibritin protein as described in Krasnykh et al. (2001. J. Virol. 75:4176-4183; which is incorporated by reference in its entirety) to allow for incorporation and display of the 117-amino acid extracellular immunoglobulin-like domain of human CD47, which interacts with CD172a. The amino-terminal segment of Ad5 fiber sequence is genetically fused with the carboxy-terminal portion of the T4 fibritin protein, followed by a linker and a six-His-containing ligand. The beginning of the third pseudorepeat of the fiber shaft domain (GNTLSQNV; SEQ ID NO:2) is joined to the fibritin sequence starting with the fragment of the insertion loop (SQN) preceding the fifth coiled-coil segment of the α-helical central domain of the fibritin (VYSRLNEIDTKQTTVESDISAIKTSI; SEQ ID NO:3). The sequence SQNV (SEQ ID NO:4) present in the native structures of both fusion partners provides a hinge between the two molecules to minimize potential structural conflicts between the β-spiral configuration of the fiber shaft and the triple α-helix of the central domain of the fibritin. A peptide linker can be incorporated between the carboxy-terminal trimerization domain (foldon) of the fibritin and the six-histidine-containing ligand to extend the ligand away from the carrier protein to facilitate binding to the target receptor.

In another embodiment to modify the Ad capsid, a minimal 21 amino acid peptide computationally designed based on hCD47-hSIRPα crystal structure, as described in Rodriguez et al. (2013. Science 339:971-975; which is incorporated by reference in its entirety). A suitable 21 amino acid peptide includes SEQ ID NO:5 (GNYTCEVTELTREGETIIELK).

In another embodiment to modify the Ad capsid, camelid antibodies generated against human CD172α protein can be incorporated into the capsid following the strategy discussed in Kaliberov et al. (2014. Lab. Invest. 94:893-905; which is incorporated by reference in its entirety). Human CD172α protein in a suitable adjuvant is used to immunize alpacas (Vicugna pacos). To identify anti-CD172α VHH, library construction, panning, phage recovery, and clone fingerprinting are performed according to methods known to those skilled in the art. The anti-CD172α VHH open reading frame (ORF) is then connected with the Ad5 fiber-T4 fibritin fusion protein as discussed above.

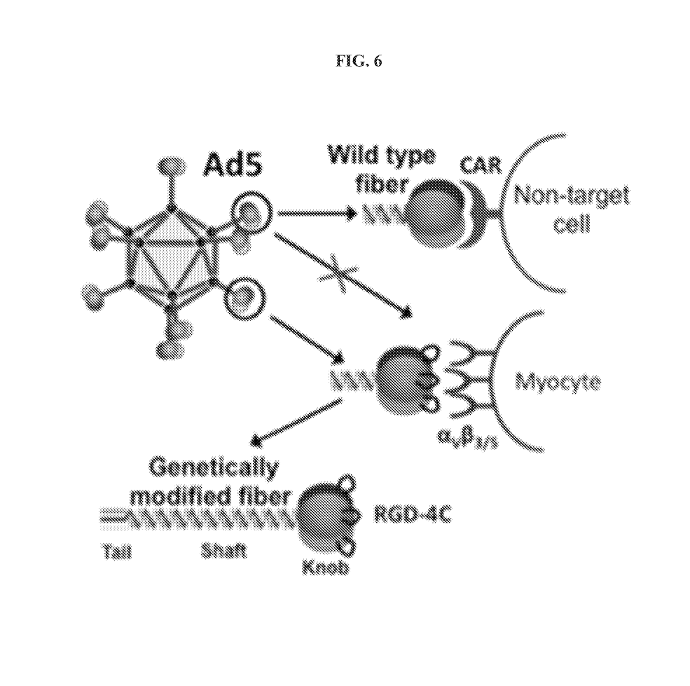

A particularly suitable modified adenovirus vector is an RGD-modified Ad5 adenovirus illustrated in FIG. 6. The RGD-modified Ad5 adenovirus is modified to direct the Ad vector to the αᵥ integrins cell surface receptor present on adult human cardiomyocytes. The RGD-modified Ad5 adenovirus achieves cell binding by including a genetic modification of the fiber knob domain to express the RGD-4C peptide, which then can bind the αᵥ integrins cell surface receptor.

Exemplary adenovirus vectors included: Ad5gfpLuc(GL), which is control Ad vector serotype 5 expressing both GFP and luciferase gene and uses coxsackievirus group B and Ad receptor (CAR) as the primary receptor and does not have any capsid modifications to enhance infectivity in cells with low CAR level (Carson, 2001 Rev. Med. Virol. 11(4):219-226; Tomko et al. 2000 Exp. Cell. Res. 255(1):47-55.); Ad5rgd-GL vector, which has the RGD-4C peptide genetically incorporated into the HI loop of the Ad5 fiber knob domain to confer CAR-independent virus tropism via αvβ5/3-integrin targeting (Dmitriev et al. 1998 J. Virol. 72(12):9706-9713); Ad5pK7-GL vector, which has a polylysine (pK7) peptide genetically incorporated into the C terminus of the Ad5 fiber knob domain to enhance virus infectivity via the use of cell-surface proteins containing polyanion motifs such as heparan sulfates (Wickham et al. 1997 J. Virol. 71(11):8221-8229); and Ad5pK7RGD-GL vector, which contains both RGD-4C and pK7 peptide motif incorporated into the Ad5 fiber knob domain (Wu et al. 2002 Hum. Gene Ter. 13(13):1647-1653); Ad5/3-GFP vector, which has Ad5 fiber knob replacement for its counterpart from Ad serotype 3 (Krasnykh et al. 1996 J. Virol. 70(10):6839-6846) that recognizes an alternative receptor, desmoglein 2 (Wang et al. 2011 Nat. Med. 17(1):96-104), which appears to be more abundantly expressed in cancer cells (Kanerva et al. 2002 Clin. Cancer Res. 8(1):275-280; Tsuruta et al. 2008 Clin. Cancer Res. 14(11):3582-3588); and Ad5PK4-GFP vector, which has a unique chimeric fiber protein that contains the tandem carbohydrate binding domains of the fiber protein of the NADC-1 strain of porcine adenovirus type 4 to augment CAR-independent tropism via targeting of cell-surface glycans (Kim et al. 2013 PLoS One 8(2):e55533; Guardado-Calvo et al. 2010 J. Virol. 84(20):10558-10568).

The composition can further include a carrier. Particularly suitable carriers can be polymers. Particularly suitable polymers can be poloxamers.

The composition can further include a protease. Particularly suitable proteases can be trypsin, collagenase, and combinations thereof.

The composition can further include a eukaryotic promoter. A particularly suitable promoter is a cytomegalovirus (CMV) promoter. Particularly suitable eukaryotic promoters include tissue-specific promoters. Particularly suitable tissue-specific promoters are cardiac tissue-specific promoters. Particularly suitable cardiac tissue-specific promoters include, for example, an α-myosin heavy chain (αMHC) promoter, a troponin-2 (Tnnt2) promoter, a myosin light chain-2v (MLC-2v), a cardiac troponin C (cTnC) promoter, a β-myosin heavy chain (βMHC) promoter, a Na⁺-Ca²⁺ exchanger H1 (NCX1H1) promoter, and combinations thereof.

The composition can further comprise an inducible promoter. Suitable inducible promoters include, for example, a tetracycline (Tet)-inducible promoter, a doxycycline (Dox)-inducible promoter, and a tamoxifen (tam)-inducible promoter. Including an inducible promoter allows for temporal control over gene expression by administration of the inducing compound. For example, two components of the Tet- (and Dox-) inducible system are the Tet repressor (TetR) and the tet operator (tetO). Both Tet and its analog doxycycline (Dox) interact with TetR and are well tolerated and widely used in mammalian systems. The Tet-ON approach can used to regulate gene expression. In the reverse Tet controlled transactivator (rtTA) or Tet-ON system, Tet or Dox binds to and induces a Tet-responsive promoter.

In particularly suitable embodiments, the human α-myosin heavy chain (αMHC) and troponin-2 (Tnnt2) promoters can be used with the Tet-inducible system to achieve an inducible cardiac tissue-specific expression of the transcription factor. Particularly suitable inducible cardiac tissue-specific systems include for example, αMHC-rtTA/tetO and Tnnt2-rtTA/tetO systems to allow for Dox-dependent myocardial-specific gene expression.

The composition can further include a small molecule. Suitable small molecules can be valproic acid, a jumonji, AT-rich interactive domain (JARID) demethylase inhibitor, a telomere binding factor β (TBFβ) inhibitor, and combinations thereof.

In another aspect, the present disclosure is directed to an adenovirus encoding a transcription factor, wherein the adenovirus comprises a modified trimeric fiber protein as described herein.

Suitable transcription factors are described herein, including combinations of transcription factors as described herein.

The adenovirus can further include a promoter as described herein. Suitable promoters include tissue-specific promoters, inducible promoters and combinations thereof as described herein.

In one embodiment, the present disclosure is directed to an RGD-modified Ad5 adenovirus vector encoding at least one transcription factor, wherein the Ad5 adenovirus vector comprises an inducible cardiac tissue-specific expression system wherein the inducible cardiac tissue-specific expression system includes a cardiac tissue specific promoter operably linked to an inducible promoter and wherein the inducible cardiac tissue-specific expression system is operably linked to at least one transcription factor. Particularly suitable inducible cardiac tissue-specific systems include for example, αMHC-rtTA/tetO and Tnnt2-rtTA/tetO systems, as described herein.

Exemplary adenovirus vectors included: Ad5gfpLuc(GL), which is control Ad vector serotype 5 expressing both GFP and luciferase gene and uses coxsackievirus group B and Ad receptor (CAR) as the primary receptor and does not have any capsid modifications to enhance infectivity in cells with low CAR level (Carson, 2001 Rev. Med. Virol. 11(4):219-226; Tomko et al. 2000 Exp. Cell. Res. 255(1):47-55.); Ad5rgd-GL vector, which has the RGD-4C peptide genetically incorporated into the HI loop of the Ad5 fiber knob domain to confer CAR-independent virus tropism via αvβ5/3-integrin targeting (Dmitriev et al. 1998 J. Virol. 72(12):9706-9713); Ad5pK7-GL vector, which has a polylysine (pK7) peptide genetically incorporated into the C terminus of the Ad5 fiber knob domain to enhance virus infectivity via the use of cell-surface proteins containing polyanion motifs such as heparan sulfates (Wickham et al. 1997 J. Virol. 71(11):8221-8229); and Ad5pK7RGD-GL vector, which contains both RGD-4C and pK7 peptide motif incorporated into the Ad5 fiber knob domain (Wu et al. 2002 Hum. Gene Ter. 13(13):1647-1653); Ad5/3-GFP vector, which has Ad5 fiber knob replacement for its counterpart from Ad serotype 3 (Krasnykh et al. 1996 J. Virol. 70(10):6839-6846) that recognizes an alternative receptor, desmoglein 2 (Wang et al. 2011 Nat. Med. 17(1):96-104), which appears to be more abundantly expressed in cancer cells (Kanerva et al. 2002 Clin. Cancer Res. 8(1):275-280; Tsuruta et al. 2008 Clin. Cancer Res. 14(11):3582-3588); and Ad5PK4-GFP vector, which has a unique chimeric fiber protein that contains the tandem carbohydrate binding domains of the fiber protein of the NADC-1 strain of porcine adenovirus type 4 to augment CAR-independent tropism via targeting of cell-surface glycans (Kim et al. 2013 PLoS One 8(2):e55533; Guardado-Calvo et al. 2010 J. Virol. 84(20):10558-10568).

In another aspect, the present disclosure is directed to a method for converting a cardiomyocyte into an induced-sinoatrial node (iSAN) cell. The method includes: infecting a cardiomyocyte with a composition comprising an adenovirus encoding a transcription factor, wherein the transcription factor comprises a β-catenin, T-box18 (Tbx18), short stature homeobox 2 (Shox2), Islet-1 (ISL1), and combinations thereof.

Particularly suitable adenovirus can be a polycistronic adenovirus. Another particularly suitable adenovirus can be a replication-defective adenovirus. The adenovirus can further include a modification of the viral capsid as described herein.

Exemplary adenovirus vectors included: Ad5gfpLuc(GL), which is control Ad vector serotype 5 expressing both GFP and luciferase gene and uses coxsackievirus group B and Ad receptor (CAR) as the primary receptor and does not have any capsid modifications to enhance infectivity in cells with low CAR level (Carson, 2001 Rev. Med. Virol. 11(4):219-226; Tomko et al. 2000 Exp. Cell. Res. 255(1):47-55.); Ad5rgd-GL vector, which has the RGD-4C peptide genetically incorporated into the HI loop of the Ad5 fiber knob domain to confer CAR-independent virus tropism via αvβ5/3-integrin targeting (Dmitriev et al. 1998 J. Virol. 72(12):9706-9713); Ad5pK7-GL vector, which has a polylysine (pK7) peptide genetically incorporated into the C terminus of the Ad5 fiber knob domain to enhance virus infectivity via the use of cell-surface proteins containing polyanion motifs such as heparan sulfates (Wickham et al. 1997 J. Virol. 71(11):8221-8229); and Ad5pK7RGD-GL vector, which contains both RGD-4C and pK7 peptide motif incorporated into the Ad5 fiber knob domain (Wu et al. 2002 Hum. Gene Ter. 13(13):1647-1653); Ad5/3-GFP vector, which has Ad5 fiber knob replacement for its counterpart from Ad serotype 3 (Krasnykh et al. 1996 J. Virol. 70(10):6839-6846) that recognizes an alternative receptor, desmoglein 2 (Wang et al. 2011 Nat. Med. 17(1):96-104), which appears to be more abundantly expressed in cancer cells (Kanerva et al. 2002 Clin. Cancer Res. 8(1):275-280; Tsuruta et al. 2008 Clin. Cancer Res. 14(11):3582-3588); and Ad5PK4-GFP vector, which has a unique chimeric fiber protein that contains the tandem carbohydrate binding domains of the fiber protein of the NADC-1 strain of porcine adenovirus type 4 to augment CAR-independent tropism via targeting of cell-surface glycans (Kim et al. 2013 PLoS One 8(2):e55533; Guardado-Calvo et al. 2010 J. Virol. 84(20):10558-10568).

Particularly suitable combinations include, for example, β-catenin+Tbx18, β-catenin+Shox2, β-catenin+ISL1, β-catenin+Tbx18+Shox2, β-catenin+Tbx18+Shox2+ISL1, β-catenin+Tbx18+ISL1, and β-catenin+Shox2+ISL1. Other suitable transcription factors include Tbx3 and canonical Wnt as described herein. As used herein, combinations of transcription factors can occur in any order when a combination of transcription factors is encoded by a single vector as described herein. In additional embodiments, related family members of these transcription factors can be used. In some embodiments, human transcription factors are particularly suitable. In other embodiments, homologs from different species are used (either in place of or in conjunction with the human transcription factor(s)). In some embodiments, the transcription factors are administered separately. In some embodiments, the transcription factors are administered at discrete time frames. In some embodiments, the transcription factors are administered in overlapping time frames. In some embodiments, a functional fragment of one transcription factor is used in conjunction with one (or more) full length transcription factors. In other embodiments, combinations of functional fragments of transcription factors are used

Particularly suitable β-catenins can be constitutively active β-catenins as described herein.

The composition can further include a eukaryotic promoter as described herein, including tissue-specific promoters, inducible promoters, and combinations thereof

The cardiomyocyte can be obtained from cardiac tissue from an intact heart, a heart slice, a cardiac explant, and an isolated cardiomyocyte. The cardiomyocyte can be an atrial cardiomyocyte and ventricular cardiomyocyte. The atrial cardiomyocyte can be right atrial cardiomyocyte and left atrial cardiomyocyte. The ventricular cardiomyocyte can be right ventricular cardiomyocyte and left ventricular cardiomyocyte.

The composition of the method can further include a protease. Particularly suitable proteases can be trypsin, collagenase, and combinations thereof.

The composition of the method can further include a carrier. Particularly suitable carriers include polymers. Particularly suitable polymers are poloxamers.

The composition of the method can further include a small molecule. Particularly suitable small molecules can be valproic acid, a JARID demethylase inhibitor, a TBFβ inhibitor, and combinations thereof.

In another aspect, the present disclosure is directed to method for treating sinus node dysfunction (SND) in an individual in need thereof. The method includes: administering to the individual a composition comprising an adenovirus encoding a transcription factor and a cardiac tissue-specific promoter.

In some embodiments, the composition can further include a carrier as described herein.

In some embodiments, the composition can further include a protease as described herein.

A particularly suitable administration method can be in situ application of the composition to a cardiac tissue. Another particularly suitable administration method can be genetic painting of the composition to a cardiac tissue. The epicardial “gene painting” technique involves application of virus locally onto the surface of the a cardiac using a polymer and dilute protease enabling a prolonged contact of the viral vector with atrial tissue, increasing gene transfer and enhancing focal gene delivery. Another particularly suitable administration method can be catheter injection of the composition to a cardiac tissue. Suitably, administration is to a site selected from the apex of the heart, right branch of the Bundle of His, the left branch of the Bundle of His, the Purkinje fibers, the inter-ventricular septum, the right ventricular free wall, the left ventricular free wall, the SA node, the AV node, and combinations thereof. Suitably, an administration site can be accessed via the right ventricle, accessed via the right atrium, and via directly accessing the heart. Accessing the administration site can be achieved by a map guided catheter injection system, by fluoroscopy guidance, by X-ray guidance, by echocardiography guidance, and by guidance using magnetic resonance imaging, for example.

Particularly suitable adenovirus can be a polycistronic adenovirus as described herein. Another particularly suitable adenovirus can be a replication-defective adenovirus as described herein. The adenovirus can further include a modification of the viral capsid as described herein. A particularly suitable adenovirus is an RGD-modified adenovirus as described herein.

Exemplary adenovirus vectors included: Ad5gfpLuc(GL), which is control Ad vector serotype 5 expressing both GFP and luciferase gene and uses coxsackievirus group B and Ad receptor (CAR) as the primary receptor and does not have any capsid modifications to enhance infectivity in cells with low CAR level (Carson, 2001 Rev. Med. Virol. 11(4):219-226; Tomko et al. 2000 Exp. Cell. Res. 255(1):47-55.); Ad5rgd-GL vector, which has the RGD-4C peptide genetically incorporated into the HI loop of the Ad5 fiber knob domain to confer CAR-independent virus tropism via αvβ5/3-integrin targeting (Dmitriev et al. 1998 J. Virol. 72(12):9706-9713); Ad5pK7-GL vector, which has a polylysine (pK7) peptide genetically incorporated into the C terminus of the Ad5 fiber knob domain to enhance virus infectivity via the use of cell-surface proteins containing polyanion motifs such as heparan sulfates (Wickham et al. 1997 J. Virol. 71(11):8221-8229); and Ad5pK7RGD-GL vector, which contains both RGD-4C and pK7 peptide motif incorporated into the Ad5 fiber knob domain (Wu et al. 2002 Hum. Gene Ter. 13(13):1647-1653); Ad5/3-GFP vector, which has Ad5 fiber knob replacement for its counterpart from Ad serotype 3 (Krasnykh et al. 1996 J. Virol. 70(10):6839-6846) that recognizes an alternative receptor, desmoglein 2 (Wang et al. 2011 Nat. Med. 17(1):96-104), which appears to be more abundantly expressed in cancer cells (Kanerva et al. 2002 Clin. Cancer Res. 8(1):275-280; Tsuruta et al. 2008 Clin. Cancer Res. 14(11):3582-3588); and Ad5PK4-GFP vector, which has a unique chimeric fiber protein that contains the tandem carbohydrate binding domains of the fiber protein of the NADC-1 strain of porcine adenovirus type 4 to augment CAR-independent tropism via targeting of cell-surface glycans (Kim et al. 2013 PLoS One 8(2):e55533; Guardado-Calvo et al. 2010 J. Virol. 84(20):10558-10568).

Particularly suitable transcription factors can be a β-catenin, T-box 18 (Tbx18), short stature homeobox 2 (Shox2), Islet-1 (ISL1, ISL LIM Homeobox 1), and combinations thereof. Particularly suitable combinations include, for example, β-catenin+Tbx18, β-catenin+Shox2, β-catenin+ISL1, β-catenin+Tbx18+Shox2, β-catenin+Tbx18+Shox2+ISL1, β-catenin+Tbx18+ISL1, and β-catenin+Shox2+ISL1. Other suitable transcription factors include, for example, T-box 3 (Tbx3) and canonical Wnt. As used herein, combinations of transcription factors can occur in any order when a combination of transcription factors is encoded by a single vector. For example, a combination of β-catenin+Tbx18 can be oriented in the vector as β-catenin-Tbx18 or Tbx18-β-catenin. In additional embodiments, related family members of these transcription factors can be used. In some embodiments, human transcription factors are particularly suitable. In other embodiments, homologs from different species are used (either in place of or in conjunction with the human transcription factor(s)). In some embodiments, the transcription factors are administered separately. In some embodiments, the transcription factors are administered at discrete time frames. In some embodiments, the transcription factors are administered in overlapping time frames. In some embodiments, a functional fragment of one transcription factor is used in conjunction with one (or more) full length transcription factors. In other embodiments, combinations of functional fragments of transcription factors are used.

Particularly suitable β-catenins can be constitutively active β-catenins as described herein.

Particularly suitable cardiac tissue-specific promoters are described herein. The adenovirus can further include inducible promoters as described herein. The adenovirus can further encode combinations of cardiac tissue-specific promoters and inducible promoters as described herein.

The composition of the method can further include a protease. Particularly suitable proteases can be trypsin, collagenase, and combinations thereof.

The composition of the method can further include a carrier. Particularly suitable carriers include polymers. Particularly suitable polymers are poloxamers.

The composition of the method can further include a small molecule. Particularly suitable small molecules can be valproic acid, a JARID demethylase inhibitor, a TBFβ inhibitor, and combinations thereof.

The dose of a viral construct to be administered is based on plaque-forming units (pfu), which is a well-established unit of measurement in the viral arts. Suitable dose ranges from about 1×10⁸ pfu to about 1×10¹⁰ pfu (in volumes ranging from about 50 microliters to about 200 microliters) are used. Higher or lower doses may be used, depending on, for example, the severity of cardiac disease or condition in the individual, the presence or absence of an electronic pacemaker, and the size of the individual's heart.

The method can result in a change in the rhythm of the heart. Suitably, the in the rhythm of the heart corresponds to a new heart rate within about 25% to about 35% of a normal heart rate.

The method can result in a reduction in the dependence of an individual on an implanted pacemaker.

In another aspect, the present disclosure is directed to a method for converting cardiac tissue to an induced-sinoatrial node. The method includes contacting the cardiac tissue with a composition comprising an adenovirus encoding a transcription factor and encoding a cardiac tissue-specific promoter.

Particularly suitable adenovirus can be a polycistronic adenovirus. Another particularly suitable adenovirus can be a replication-defective adenovirus.

The adenovirus can further include a modification of the viral capsid. Particularly suitable modifications can be an RGD sequence; polylysine residues; a serotype Ad5 knob and a serotype Ad3 knob; a binding motif that specifically binds to a cell-surface glycan; an antibody that specifically binds to a cardiomyocyte cell-surface receptor; a CD47 peptide; and combinations thereof. A particularly suitable CD47 peptide can be a humanized CD47 peptide.

Exemplary adenovirus vectors included: Ad5gfpLuc(GL), which is control Ad vector serotype 5 expressing both GFP and luciferase gene and uses coxsackievirus group B and Ad receptor (CAR) as the primary receptor and does not have any capsid modifications to enhance infectivity in cells with low CAR level (Carson, 2001 Rev. Med. Virol. 11(4):219-226; Tomko et al. 2000 Exp. Cell. Res. 255(1):47-55.); Ad5rgd-GL vector, which has the RGD-4C peptide genetically incorporated into the HI loop of the Ad5 fiber knob domain to confer CAR-independent virus tropism via αvβ5/3-integrin targeting (Dmitriev et al. 1998 J. Virol. 72(12):9706-9713); Ad5pK7-GL vector, which has a polylysine (pK7) peptide genetically incorporated into the C terminus of the Ad5 fiber knob domain to enhance virus infectivity via the use of cell-surface proteins containing polyanion motifs such as heparan sulfates (Wickham et al. 1997 J. Virol. 71(11):8221-8229); and Ad5pK7RGD-GL vector, which contains both RGD-4C and pK7 peptide motif incorporated into the Ad5 fiber knob domain (Wu et al. 2002 Hum. Gene Ter. 13(13):1647-1653); Ad5/3-GFP vector, which has Ad5 fiber knob replacement for its counterpart from Ad serotype 3 (Krasnykh et al. 1996 J. Virol. 70(10):6839-6846) that recognizes an alternative receptor, desmoglein 2 (Wang et al. 2011 Nat. Med. 17(1):96-104), which appears to be more abundantly expressed in cancer cells (Kanerva et al. 2002 Clin. Cancer Res. 8(1):275-280; Tsuruta et al. 2008 Clin. Cancer Res. 14(11):3582-3588); and Ad5PK4-GFP vector, which has a unique chimeric fiber protein that contains the tandem carbohydrate binding domains of the fiber protein of the NADC-1 strain of porcine adenovirus type 4 to augment CAR-independent tropism via targeting of cell-surface glycans (Kim et al. 2013 PLoS One 8(2):e55533; Guardado-Calvo et al. 2010 J. Virol. 84(20):10558-10568).

Particularly suitable transcription factors can be a β-catenin, T-box 18 (Tbx18), short stature homeobox 2 (Shox2), Islet-1 (ISL1, ISL LIM Homeobox 1), and combinations thereof. Particularly suitable combinations include, for example, β-catenin+Tbx18, β-catenin+Shox2, β-catenin+ISL1, β-catenin+Tbx18+Shox2, β-catenin+Tbx18+Shox2+ISL1, β-catenin+Tbx18+ISL1, and β-catenin+Shox2+ISL1. Other suitable transcription factors include, for example, T-box 3 (Tbx3) and canonical Wnt. As used herein, combinations of transcription factors can occur in any order when a combination of transcription factors is encoded by a single vector. For example, a combination of β-catenin+Tbx18 can be oriented in the vector as β-catenin-Tbx18 or Tbx18-β-catenin. In additional embodiments, related family members of these transcription factors can be used. In some embodiments, human transcription factors are particularly suitable. In other embodiments, homologs from different species are used (either in place of or in conjunction with the human transcription factor(s)). In some embodiments, the transcription factors are administered separately. In some embodiments, the transcription factors are administered at discrete time frames. In some embodiments, the transcription factors are administered in overlapping time frames. In some embodiments, a functional fragment of one transcription factor is used in conjunction with one (or more) full length transcription factors. In other embodiments, combinations of functional fragments of transcription factors are used.

Particularly suitable β-catenins can be constitutively active β-catenins as described herein.

The cardiac tissue can be a heart slice, an isolated cardiomyocyte, a cardiac explant, an atrial tissue slice and a ventricle tissue slice. Suitable atrial tissue slices can be, for example, Sinus node, crista terminalis, free walls, appendages, atrioventricular junction, atrioventricular node, and combinations thereof. Suitable ventricular tissue slices can be, for example, atrioventricular junction, His bundle, bundle branches, Purkinje cells, free walls, septum, outflow tract, and combinations thereof.

The composition of the method can further include a protease. Particularly suitable proteases can be trypsin, collagenase, and combinations thereof.

The composition of the method can further include a carrier. Particularly suitable carriers include polymers. Particularly suitable polymers are poloxamers.

The composition of the method can further include a small molecule. Particularly suitable small molecules can be valproic acid, a JARID demethylase inhibitor, a TBFβ inhibitor, and combinations thereof.

The composition can further include a eukaryotic promoter as described herein, including tissue-specific promoters, inducible promoters, and combinations thereof.

In another aspect, the present disclosure is directed to a DNA delivery system comprising a polynucleotide encoding a transcription factor, wherein the transcription factor comprises a β-catenin, T-box18 (Tbx18), short stature homeobox 2 (Shox2), Islet-1 (ISL1), and combinations thereof.

A particularly suitable DNA delivery system includes a viral vector. Particularly suitable viral vectors include, for example, adenovirus, adeno-associated virus, lentivirus, retrovirus, Highlands J virus (HJV), human immunodeficiency virus (HIV), and Herpes simplex viruses (HSV). In embodiments wherein more than one transcription factor is administered, the transcription factors can optionally be included in different viral vectors. Alternatively, in some embodiments, multiple transcription factors can be included in a single viral vector.

Another particularly suitable DNA delivery system includes a non-viral vector. Particularly suitable non-viral vectors include, for example, liposomal vectors, a cationic polymers, and DNA binding polymers.

Another particularly suitable DNA delivery system includes naked DNA.

Particularly suitable transcription factors can be a β-catenin, T-box 18 (Tbx18), short stature homeobox 2 (Shox2), Islet-1 (ISL1, ISL LIM Homeobox 1), and combinations thereof. Particularly suitable combinations include, for example, β-catenin+Tbx18, β-catenin+Shox2, β-catenin+ISL1, β-catenin+Tbx18+Shox2, β-catenin+Tbx18+Shox2+ISL1, β-catenin+Tbx18+ISL1, and β-catenin+Shox2+ISL1. Other suitable transcription factors include, for example, T-box 3 (Tbx3) and canonical Wnt. As used herein, combinations of transcription factors can occur in any order when a combination of transcription factors is encoded by a single vector. For example, a combination of β-catenin+Tbx18 can be oriented in the vector as β-catenin-Tbx18 or Tbx18-β-catenin. In additional embodiments, related family members of these transcription factors can be used. In some embodiments, human transcription factors are particularly suitable. In other embodiments, homologs from different species are used (either in place of or in conjunction with the human transcription factor(s)). In some embodiments, the transcription factors are administered separately. In some embodiments, the transcription factors are administered at discrete time frames. In some embodiments, the transcription factors are administered in overlapping time frames. In some embodiments, a functional fragment of one transcription factor is used in conjunction with one (or more) full length transcription factors. In other embodiments, combinations of functional fragments of transcription factors are used.

In another aspect, the present disclosure is directed to a cardiac slice culture system comprising: a cardiac tissue slice; and an incubation apparatus.

Suitable cardiac tissue slice can be, for example, an atrial tissue slice, a ventricle tissue slice, and combinations thereof, as described herein. The cardiac tissue slice can be, for example, a human cardiac tissue slice, a porcine cardiac tissue slice, a mouse cardiac tissue slice, a rat cardiac tissue slice, a rabbit cardiac tissue slice a guinea pig cardiac tissue slice, a bovine cardiac tissue slice, and an equine cardiac tissue slice.

The cardiac tissue slice is cultured at a liquid-air interface.

The cardiac tissue slice ranges from about 200 μm thick to about 400 μm thick.

The cardiac slice culture system can further include an electrode to provide electrical stimulation to the cardiac tissue slice. The cardiac slice culture system can further include a recording circuit.

In another aspect, the present disclosure is directed to a method for evaluating a candidate cardiac therapy. The method includes providing a cardiac tissue slice; contacting the cardiac tissue slice with a candidate cardiac therapy; and analyzing the cardiac tissue slice.

Candidate cardiac therapies include, for example, biological therapies, pharmaceutical (drug) therapies, and combinations thereof. Candidate cardiac therapies can also include non-therapeutic compositions such as, for example, carriers (e.g., pharmaceutical carriers), biological and drug vehicles, reagents and combinations thereof, which are sought to be analyzed for its effect on cardiac tissue.

The cardiac tissue slice is suitably sliced tangential to the endocardium. The cardiac tissue slice can be obtained using a high precision (1 μm) vibrating microtome. Suitable slice thickness ranges from about 200 μm to about 400 μm.

The incubation apparatus can be any culture apparatus known to those skilled in the art. A particularly suitable incubation apparatus includes, for example, a 6-well culture plate including Transwell inserts.

The cardiac tissue slice is suitably cultured at a liquid-air interface.

Suitable cardiac tissues are described herein.

The cardiac tissue slice can be analyzed by methods known to those skilled in the art. For example, the cardiac tissue slice can be analyzed by microscopy such as, for example, bright field microscopy, fluorescent microscopy, confocal microscopy, electron microscopy, Western blot analysis, enzyme-linked immunosorbent assay (ELISA), immunoprecipitation, immunohistology, in situ hybridization, Northern blot analysis, Southern blot analysis, amplification, and combinations thereof. The cardiac tissue slice can further be analyzed using output measures to assess viability of slices during culture including, for example, optical recordings of APs and CaTs, quantification of restitution properties of APD, CaT and conduction velocity using optical mapping techniques, multi-electrode array (MEA) recordings, intracellular microelectrode recordings, and immunohistological criteria.

Suitable culture media for culturing the cardiac tissue slice include, for example, Tyrode's medium (126.7 mM NaCl, 5.4 mM KCl, 1.05 mM MgCl₂, 1.8 mM CaCl₂, 0.42 mM NaH₂PO₄, 22 mM NaHCO₃, 5 mM glucose), serum-based medium (DMEM/F12, 20% knockout serum replacement, 1% non-essential amino acids, 2 mM L-glutamine, 0.1% (3-mercaptoethanol, 0.1% penicillin/streptomycin), serum-free medium (Medium 199, 1% penicillin/streptomycin, 1% insulin, transferrin, and selenium supplement), and lipid-based medium (DMEM, 10 mM galactose, 100 μM oleic acid, 50 μM palmitic acid).

Optionally, oxygen can be constantly bubbled into the culture medium to increase the oxygen concentration in the medium and to allow for improved oxygen diffusion into the cardiac tissue slice.

The cardiac tissue slice culture can further include continuous pacing of the slice. Pacing can be achieved using PowerLab (commercially available from ADInstrument) to drive the pacing electrode and record local electrocardiograms. An alternative continuous pacing system using a custom electrical stimulation and recording circuit can be incorporated into the culture system as described in Xu et al. (2014. Nat. Commun. 5:3329); Gutbrod et al. (2014. Frog. Biophys. Mol. Biol. 115:244-251); and Xu et al. (2015. Adv. Mater. 27:1731-1737). Platinum-iridium pacing electrodes can be incorporated into the slice culture system to provide continuous pacing, similar to settings that have been used for pacing ventricular slices.

A multi-electrode array (MEA) system can optionally be incorporated into the slice culture system by using MEA wells to provide high-density electrical mapping. MEA recording can allow for daily monitoring of functional parameters of the cardiac slices.

### EXAMPLES

### Example 1

In this Example, ectopic activation of canonical Wnt signaling within ventricular myocardium was investigated.

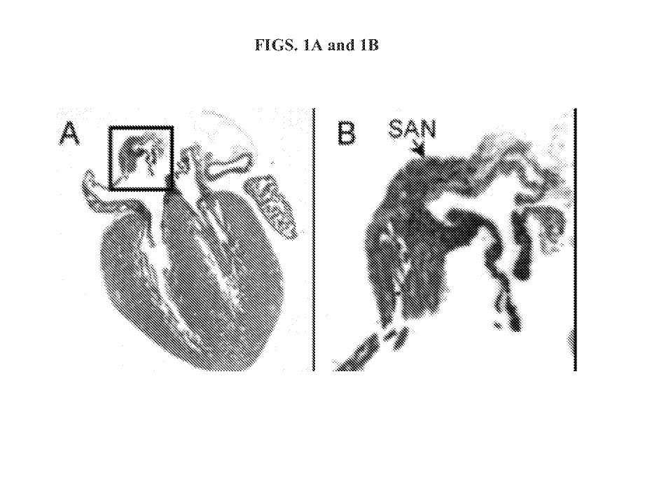

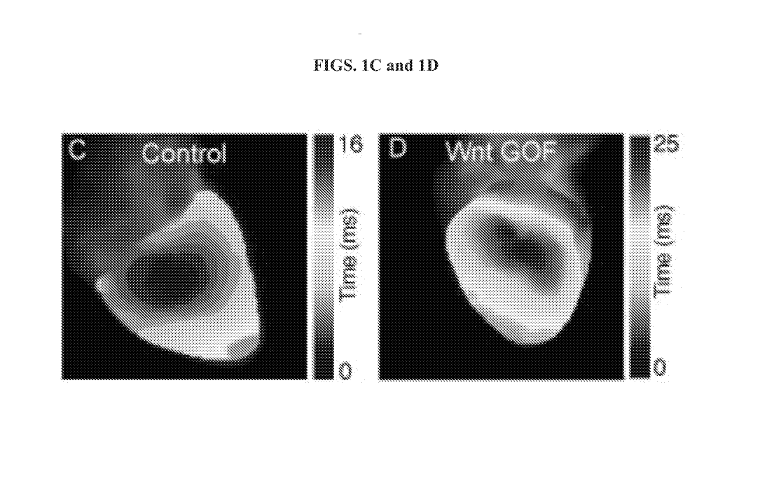

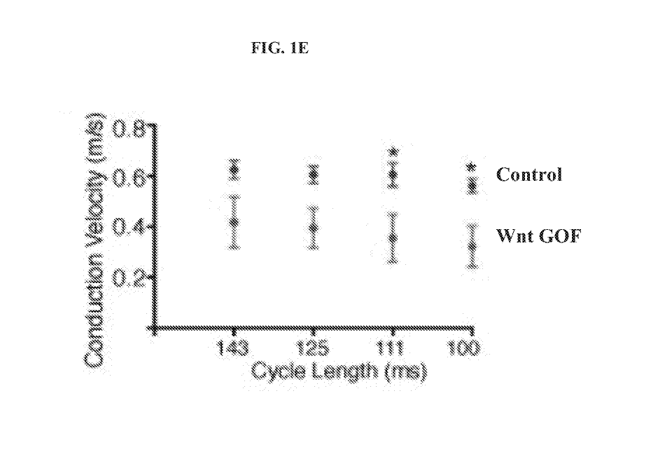

As demonstrated in FIGS. 1A and 1B, the canonical Wnt reporter Axin2^{LacZ} was expressed within the newborn SAN in the atrioventricular canal (AVC) of mice hearts. As shown in FIGS. 1C and 1D, optical mapping of electrical activation patterns in ventricles of control littermates and Wnt gain-of-function (“GOF”) adult mice demonstrated that in sinus rhythm, the PR interval and QRS duration were significantly prolonged in Wnt GOF mice (FIG. 1D) as compared to littermate control mice (FIG. 1C) (Gillers et al. 2015. Circ. Res. 116:398-406). Total epicardial activation time was significantly prolonged in Wnt GOF mice (4.4±0.4 ms in control versus 11.5±1.0 ms in Wnt GOF). Programmed electrical stimulation of the epicardial surface of the left ventricle and right ventricle further demonstrated a significant decrease in epicardial conduction velocity in both ventricles of Wnt activated mice. As shown in FIG. 1E, left ventricle longitudinal conduction velocity of Wnt GOF mice was slower during stimulation at each cycle length and the difference between the 2 genotypes became larger at faster pacing rates (111 and 100 ms cycle lengths). Right ventricle longitudinal conduction velocity of Wnt GOF mice was also slower. Unexpectedly, the right ventricle was more severely affected than the left ventricle. These results indicated decremental conduction, which is a property of AVC and AV nodal tissues.

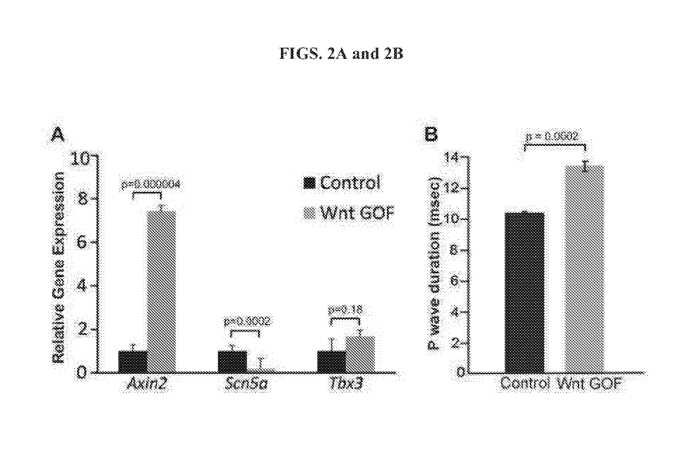

Additional data indicated that Wnt activation also regulates electrical properties in the adult heart. Canonical Wnt signaling was transiently activated in adult atria of Wnt GOF mice (αMHC-rtTA; TRE-Cre; Ctnnb1^{flox(ex3)+/}) and littermate controls by feeding mice doxycline chow. As shown in FIG. 2A, gene expression analysis in left atrial tissue by qRT-PCR demonstrated robust activation of Axin2, a direct target of canonical Wnt signaling, and down-regulation of Scn5a encoding Nav1.5. As shown in FIG. 2B, transient Wnt activation in atrial myocardium significantly slowed atrial conduction, as evidenced by prolongation of p wave duration.

### Example 2

In this Example, acute and chronic therapeutic effects in a human cardiac slice culture were investigated.

Non-failing donor hearts that were rejected for transplantation were cardioplegically arrested and cooled in the operating room following the same procedure accepted for heart transplantation. Cardiac tissue that was promptly delivered to the research laboratory, where it was sliced tangential to the endocardium using a high precision (1 μm) vibrating microtome, transferred to a custom-designed incubation apparatus, and cultured at a liquid-air interface in 6-well culture plates using Transwell inserts. Slices were cultured in M-199 supplemented with 1×ITS (Insulin, Transferrin, Selenium), 10 mM 2,3-butanedione monoxime, and 2% penicillin-streptomycin in a 37° C. incubator with humidified air with 5% CO₂. Culture medium was changed daily. Vital tissue slices from adult myocardium prepared in this manner have previously been cultured for up to 28 days with high cellular viability and maintenance of expression of the major ion channels and action potential characteristics.

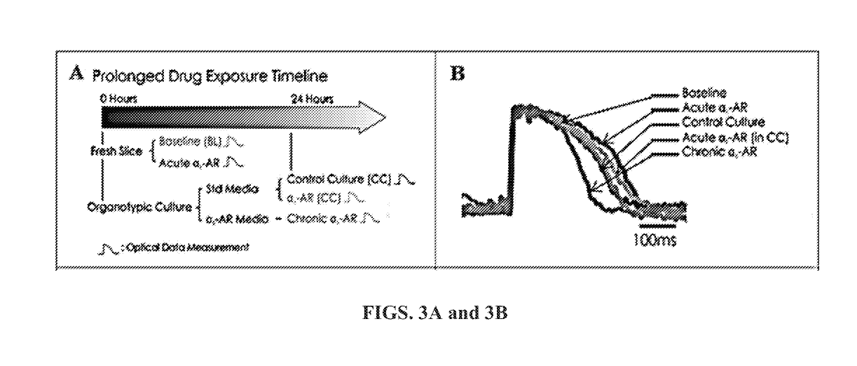

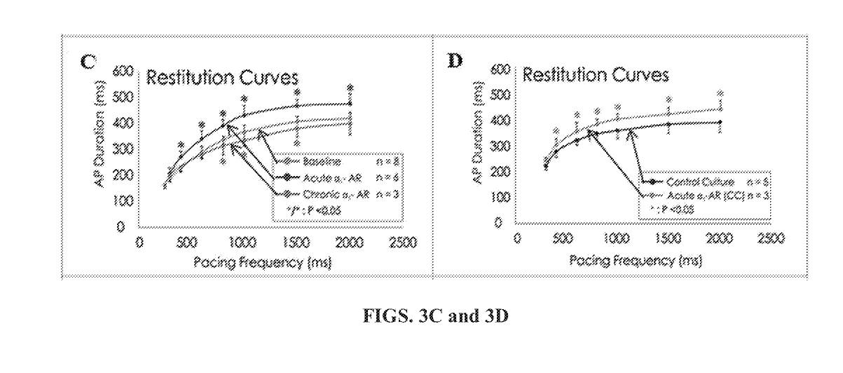

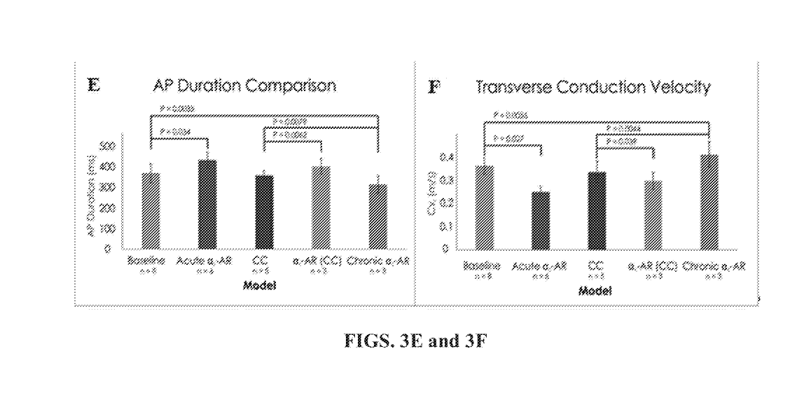

Conduction velocity, action potential and calcium transient restitution properties, and responsiveness to drugs were preserved in ventricular slices cultured for 2 days. FIGS. 3A-3F show data from an investigation of α₁-adrenergic receptor stimulation with phenylephrine, demonstrating the utility of the cardiac slice culture system for monitoring acute and chronic therapeutic effects on electrical parameters.

FIG. 3A illustrates a timeline of the experimental protocol in human ventricular slices. Significantly different effects on cardiac slice electrophysiology were observed when phenylephrine was applied acutely (15 minutes) versus chronically (24 hours) (FIG. 3B). Restitution curves shown in FIGS. 3C and 3D for each condition demonstrated that acute α₁-AR stimulation at baseline as well as treatment after one day in culture shifted the AP restitution curve upwards significantly, while chronic α₁-AR stimulation shifted the AP restitution curve downwards. No effect of culture on restitution properties was noted. FIG. 3E shows statistical difference calculated for AP duration between each condition at 1 Hz (60 BPM) pacing frequency, demonstrating similar APD lengthening with acute phenylephrine treatment at baseline, as well as after slices were cultured for one day. Chronic phenylephrine treatment resulted in APD shortening. No effect of culture on APD was noted. FIG. 3F shows that acute α₁-AR stimulation at baseline and after culture for one day significantly decreased CV, while chronic α₁-AR stimulation increased CV. There was no effect of culture on CV.

These results demonstrate that the slice culture model provides a unique system for testing human cardiac physiology in acute versus chronic settings.

### Example 3

In this Example, action potential (AP) and calcium transient (CaT) were analyzed in human atrial slices from the sino-atrial node (SAN) region.

Tissue slices can be sustained for much longer times through superfusion due to their thickness below the diffusion limit of oxygen and nutrients. As a first step toward culturing human atrial tissue, several physiologic parameters were systematically measured from acutely prepared human atrial and ventricular slices procured from various regions throughout the heart.

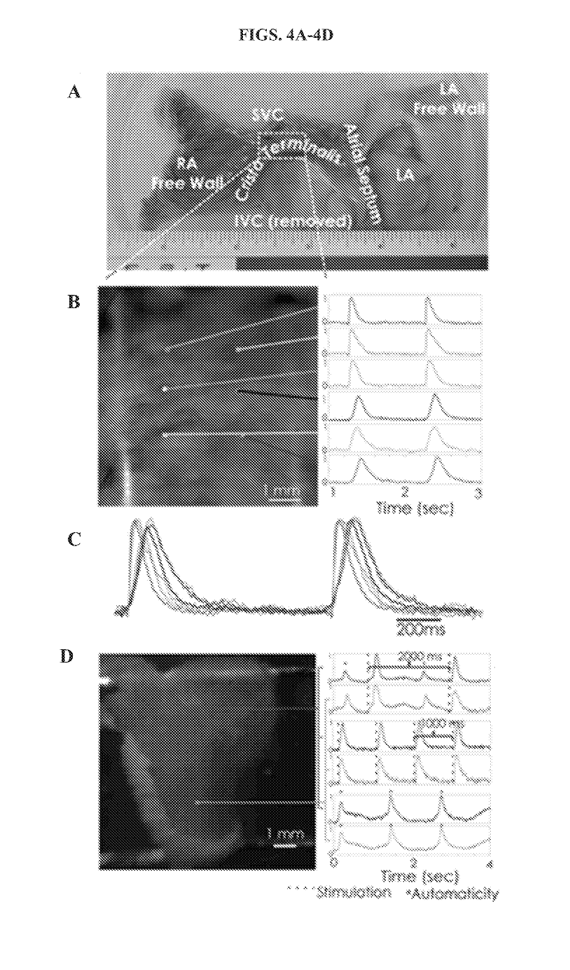

FIGS. 4A-4D illustrate optically mapping either Vₘ or [Ca²⁺] in superfused atrial and SAN tissue without the need for coronary perfusion. Slices from the crista terminalis region bordering the superior vena cava (SVC) are illustrated in FIG. 4A. FIG. 4A is a photograph of an intact atrial preparation prior to sectioning. Boxed region of the crista terminalis bordering SAN is shown in FIG. 4B. In contrast to the ventricular optical APs and CaTs, which were relatively homogeneous throughout each slice (not shown), both the optical action potentials and calcium transients from the crista terminalis/SAN region exhibited significant heterogeneity in morphology and duration due to the slice containing both SAN and working atrial tissue. Two distinctly different upstroke velocities and morphologies were observed: SAN cells exhibited slow upstrokes and longer duration of action potentials, while the working atrial myocardium exhibited faster upstrokes and shorter waveforms. FIG. 4C illustrates CaT recorded from distinct atrial regions demonstrating heterogeneity in the CaT morphology. Faster Ca release and uptake were observed in regions of contractile atrial myocardium (traces shifted to the left), while slower CaT upstroke and recovery were recorded in the sinus node area (traces shifted to the right).

To determine the inherent automaticity of slices from the SAN region containing both nodal and working atrial tissue, optical action potentials were recorded during pacing at a long cycle length of 2000 ms (30 beats per minute/BPM). FIG. 4D shows both capture of paced beats as well as beats originating from the competing SAN automaticity which occurred at approximately 1 second cycle length (60 BPM). Without stimulation, only spontaneous SAN automaticity was observed. This spontaneous activity was reflective of SAN automaticity and not damaged myocardium because ventricular and atrial muscle slices lacking the SAN region were electrically quiescent (not shown). These results indicate a physiologic gradient in the heart, which is further supported by physiological data provided by optical mapping of the SAN excitation during superior, bifocal and inferior atrial breakthrough patterns that demonstrate a predilection for origination of spontaneous electrical activity within distinct regions within the heart (see, Fedorov et al. 2010. J. Am. College Cardiol. 56:1386-1394, FIG. 2). This physiologic gradient correlates with a similar transcriptional gradient within the heart (Rentschler et al. 2001. Development 128(10):1785-92, (see, FIG. 1); Wiese et al. 2009. Circulation Research 104:388-397). Moreover, cells nearest the conduction system have a predilection for more robust conversion to conduction-like cells (Rentschler et al. 2012. Circulation 126(9):1058-66). Therefore, targeted transcription factor delivery to specific regions of the heart according to the present disclosure can take advantage of this transcriptional gradient for converting cardiomyocytes of the heart by administering combinations of transcriptional factors specific to the transcriptional factor unit within a particular heart region.

### Example 4

In this Example, adenoviral-mediated gene expression in adult human ventricular slices was investigated.

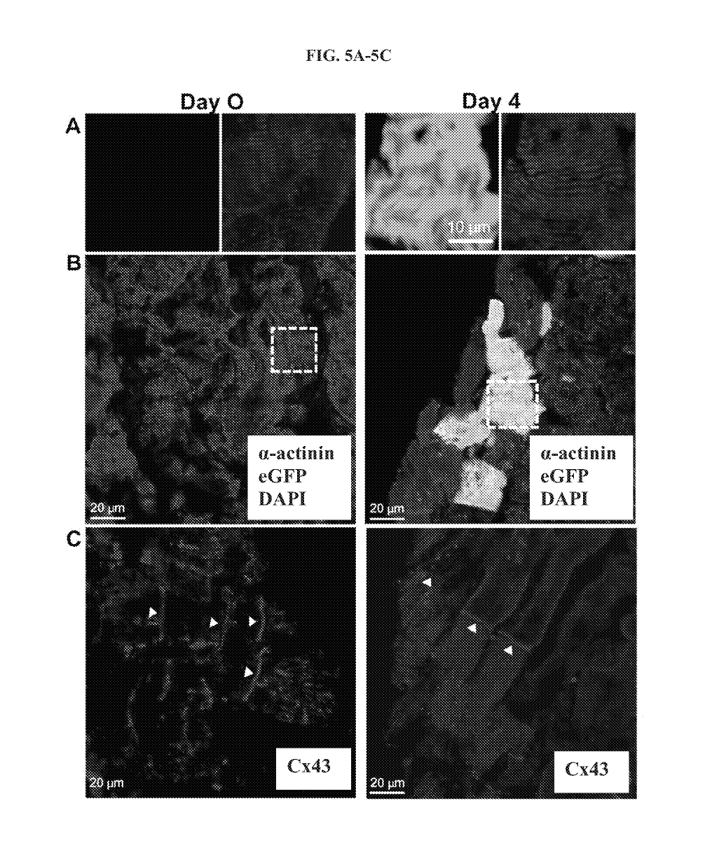

Human ventricular slices were infected with adenovirus engineered to express eGFP driven by a CMV promoter. Direct monitoring of green fluorescence was used to monitor viral transduction and transgene expression at multiple time points after viral application up to 4 days (FIG. 5A-5C). Panels in FIG. 5A show boxed regions from FIG. 5B at higher magnification with green (left) and red (right) channels separately. FIG. 5B shows merged images of the green (showing eGFP), red (showing α-actinin, cardiomyocytes) and blue (showing DAPI staining to visualize nuclei) channels. Expression of virally-encoded eGFP persisted after 4 days in culture (Day 4) and α-actinin staining demonstrated preservation of the sarcomeric structure throughout prolonged culture. FIG. 5C shows immunohistochemical staining for Connexin 43, the main gap junction protein isoform expressed in human ventricular tissue, demonstrates maintenance of well-organized gap junctions at the intercalated disc throughout prolonged culture (white arrowheads).

### Example 5

In this Example, adenoviral gene therapy vectors with enhanced transduction of human myocardium were developed.

Advances in molecular virology have enabled the genetic manipulation of viruses, establishing the concept of using recombinant viral vectors to achieve higher efficiency and more specific transduction of human cardiomyocytes. Ad serotype 5 (Ad5) cellular entry is mediated by distinct binding and internalization events: the knob domain of trimeric fiber protein initiates attachment through interactions with coxsackie virus and Ad receptor (CAR), while internalization is mediated by interactions between αᵥβ3 and αᵥβ5 integrins and the arginine-glycine-aspartic acid (RGD) motif within the penton protein loop (see FIG. 6). Though CAR is expressed on cultured cells, its expression wanes rapidly after birth in organs including the heart, and its low abundance may render human myocardium resistant to CAR-dependent virus.

To confer Ad5 vectors a CAR-independent tropism, a genetic targeting platform based on key fiber knob modifications was used. Several capsid-modified Ad5 vectors designed to incorporate targeting ligands within the fiber knob domain were compared to standard Ad5 control vector using the organotypic slice cultures. Examples of fiber knob modifications that were tested include: addition of a cysteine-constrained RGD-4C (CDCRGDCFC, SEQ ID NO:1) peptide that binds αᵥ integrins expressed on human cardiomyocytes with high affinity; addition of polylysine residues that adhere to cell-surface proteins containing polyanion motifs such as heparin sulfate receptors; Ad5/3 vector containing chimeric fibers composed of the tail and shaft domains of Ad5 with the knob domain of Ad serotype 3; and chimeric fiber modifications enabling targeting of specific cell-surface glycans. These genetic modifications of fiber knob domain have been previously demonstrated to provide enhanced virus infectivity in CAR-deficient tissues (Beatty and Curiel, 2012, Chapter two—Adenovirus strategies for tissue-specific targeting. Adv. Cancer Res. 115:39-67).

Exemplary adenovirus vectors included: Ad5gfpLuc(GL), which is a control Ad vector serotype 5 expressing both GFP and luciferase gene and uses coxsackievirus group B and Ad receptor (CAR) as the primary receptor and does not have any capsid modifications to enhance infectivity in cells with low CAR level (Carson, 2001 Rev. Med. Virol. 11(4):219-226; Tomko et al. 2000 Exp. Cell. Res. 255(1):47-55.); Ad5rgd-GL vector (SEQ ID NO:6), which has the RGD-4C peptide genetically incorporated into the HI loop of the Ad5 fiber knob domain to confer CAR-independent virus tropism via αvβ5/3-integrin targeting (Dmitriev et al. 1998 J. Virol. 72(12):9706-9713); Ad5pK7-GL vector (SEQ ID NO:7), which has a polylysine (pK7) peptide genetically incorporated into the C terminus of the Ad5 fiber knob domain to enhance virus infectivity via the use of cell-surface proteins containing polyanion motifs such as heparan sulfates (Wickham et al. 1997 J. Virol. 71(11):8221-8229); and Ad5pK7RGD-GL vector, which contains both RGD-4C and pK7 peptide motif incorporated into the Ad5 fiber knob domain (Wu et al. 2002 Hum. Gene Ter. 13(13):1647-1653); Ad5/3-GFP vector, which has Ad5 fiber knob replacement for its counterpart from Ad serotype 3 (Krasnykh et al. 1996 J. Virol. 70(10):6839-6846) that recognizes an alternative receptor, desmoglein 2 (Wang et al. 2011 Nat. Med. 17(1):96-104), which appears to be more abundantly expressed in cancer cells (Kanerva et al. 2002 Clin. Cancer Res. 8(1):275-280; Tsuruta et al. 2008 Clin. Cancer Res. 14(11):3582-3588); and Ad5PK4-GFP vector, which has a unique chimeric fiber protein that contains the tandem carbohydrate binding domains of the fiber protein of the NADC-1 strain of porcine adenovirus type 4 to augment CAR-independent tropism via targeting of cell-surface glycans (Kim et al. 2013 PLoS One 8(2):e55533; Guardado-Calvo et al. 2010 J. Virol. 84(20):10558-10568).

The indicated infectivity-enhanced vectors were compared with standard Ad5 with respect to human cardiomyocyte tropism. Cardiac slices were placed on Transwell inserts during recovery, followed by treatment with collagenase to increase the depth of viral tissue penetration and achieve greater virus transduction. 15 μL of Type 2 collagenase (Worthington Biochemical Corp., Lakewood, N.J.) reconstituted at 250 units/mL in slice culture medium was pipetted onto each slice and incubated for 20 minutes. The slices were subsequently washed, followed by addition of 1.1 mL of culture medium to the Transwell. Ad5-eGFP (total of 1.11e10 viral particles diluted in a total volume of 12 μL PBS) encoding eGFP driven by the cytomegalovirus promoter was pipetted onto each slice to cover completely, and incubated on the slice for 12 hours. Slices are serially monitored for detection of virally-encoded eGFP expression.

Data demonstrated that eGFP expression commenced prior to 24 hours post-transduction, with expression levels increasing up to 48 hours, and remaining steady up to 4 days in culture (FIG. 4). Ad5 transduces few α-actinin⁺ cardiomyocytes, as well as many non-cardiomyoyctes (FIG. 6). Much more robust transgene expression was achieved in human cardiomyocytes using Ad5-RGD, where initial viral targeting is directed towards αᵥ integrins, when compared with Ad5 at the same multiplicity of infection (FIG. 6).

On this basis, Ad5-RGD vectors were engineered to express transcription factors, including Tbx18, β-catenin, and Shox2. Specifically, the genome of the Ad5-RGD vector was constructed to encode Tbx18, β-catenin, or Shox2 genes under transcriptional control of an immediate early enhancer and promoter from human cytomegalovirus (CMV) in place of the deleted early E1 region. Exemplary Ad5-RGD vectors are provided herein as Ad5-RGD and Ad5-RGDpK7. To achieve simultaneous expression of multiple transcription factors, a mechanism that relies on the self-cleaving 2A peptide was employed to efficiently produce multiple proteins from one encoded polypeptide. For example, the expression cassette was constructed to contain the single open reading frame (ORF) encoding Tbxl 8 and β-catenin separated by a furin recognition site followed by thosea asigna virus 2A peptide with the GSG linker and cloned under the CMV promoter control using the E1 shuttle plasmid. The E3 shuttle plasmid was constructed to incorporate a CMV promoter-driven eGFP reporter gene in place of nonessential early E3 region and used for homologous recombination with Ad5-RGD genome by cotransforming E. coli strain, BJ5183 as described in (Kreuzberg et al. 2006. Trends Cardiovasc Med 16:266-272). The selected viral genome plasmid was recombined with E1 shuttle plasmids constructed to encode Tbx18, β-catenin, or Shox2 transcription factors either alone or in combination to generate the genome of Ad5-RGD-Tbx18, Ad5-RGD-β-catenin, Ad5-RGD-Shox2, Ad5-RGD-Tbx18-β-catenin, Ad5-RGD-β-catenin-Shox2, and Ad5-RGD-Tbx18-Shox2 vectors, each containing the CMV-eGFP cassette in E3 region. The Ad5-RGD genome lacking transcription factors while containing CMV promoter without transgene in E1 and CMV-eGFP reporter gene in E3 region was generated to make a control vector. Each constructed plasmid containing the recombinant Ad genome was analyzed by PCR and then retransformed into E. coli strain, DH10B to be amplified and validated by restriction enzyme analysis. The resultant plasmids were digested with Pad restriction enzyme to release the constructed viral genomes prior to transfecting HEK293 cells to rescue replication incompetent E1/E3 deleted Ad5-RGD vector derivatives encoding transcription factors. The rescued viruses were up-scaled in HEK293 cells, purified by double CsCl gradient ultracentrifugation and dialysed against phosphate-buffered saline with 10% glycerol. Final aliquots of virus were analyzed spectrophotometrically by measuring the O.D. at 260 nm to determine viral particle titer and stored at −80° C. until use. The expression of Tbx18, β-catenin, and Shox2 was assessed by Western blot following infection of CAR-deficient U118 cells and their derivative, U118CAR cells, stably expressing high levels of CAR. The magnitude of infectivity and gene transfer enhancement achieved by Ad5-RGD-Tbx18, Ad5-RGD-β-catenin, Ad5-RGD-Tbx18-β-catenin, etc. vectors in CAR-deficient cells were determined based on the number of infected cells expressing eGFP reporter using epifluorescent microscopy, as compared to previously generated Ad5 and Ad5-RGD control vectors used at the same multiplicity of infection (number of viral particles per cell). Enhanced expression in human cardiomyocytes was confirmed using the slice culture methodology. The surface of ventricular slices were treated with 1.1 E10 viral particles of Ad5 or Ad5-RGD encoding EGFP after 48 hours in culture.

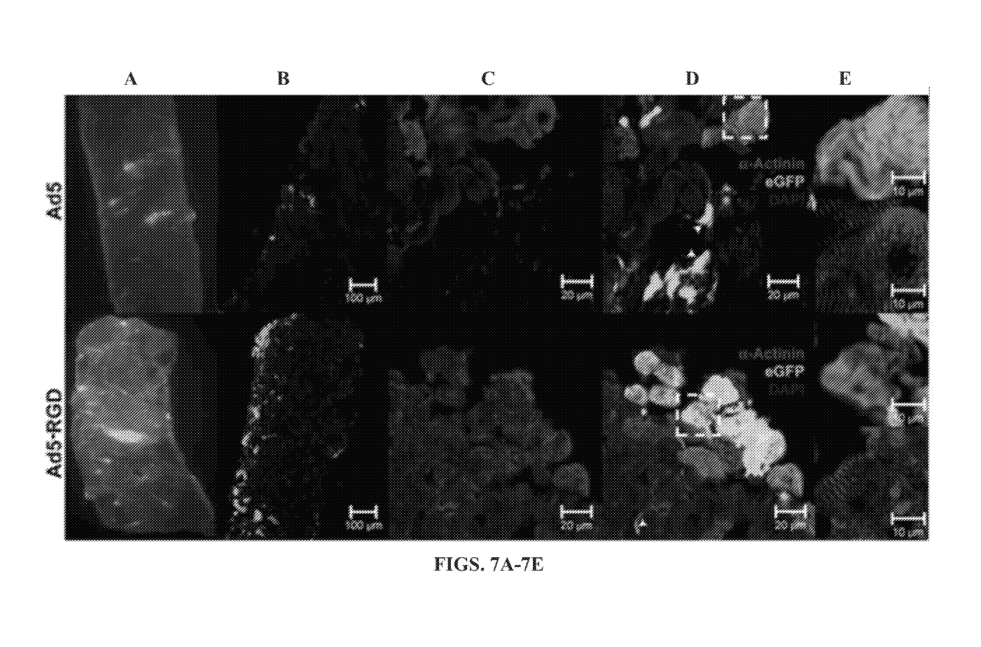

As shown in FIGS. 7A-7E, RGD-modified Ad5 improved tropism for human cardiomyocytes as compared to unmodified Ad5 after 48 hours in culture (FIG. 7A). Histologic sections through the slices demonstrate virally-transduced EGFP cells near the culture surface, co-staining with α-actinin delineates cardiomyocytes (FIG. 7B). FIG. 7C shows a higher magnification of the region from FIG. 7B demonstrating α-actinin (left panel). FIG. 7D shows α-actinin, EGFP, and DAPI co-staining. Ad5 transduced a low number of α-actinin⁺ cardiomyocytes, as well as a significant number of non-cardiomyoyctes (white arrowheads) (upper panel), while Ad5-RGD greatly enhanced the number of EGFP+ cardiomyocytes when applied at the same viral titer (lower panel). FIG. 7E shows a higher magnification of the boxed region in FIG. 7D demonstrating EGFP (top panel) and α-actinin within cardiomyocytes (lower panel), which retained a well-preserved sarcomeric structure throughout prolonged culture.

These results indicate that the Ad5-RGD vectors allow for achieving a higher efficiency and more specific transduction of human cardiomyocytes, as well as permitting use lower viral titers and evading off-target effects.

### Example 6

In this Example, human organotypic cardiac slices were analyzed for high throughput analysis of candidate human heart therapies.

Heart Tissue Acquisition

Adult human hearts were procured either as failing human hearts from Barnes-Jewish Hospital or donor human hearts from Mid-America transplant services. Explanted hearts were cardioplegically arrested via high potassium solution (in mmol/L: NaCl 110, CaCl₂ 1.2, KCl 16, MgCl₂ 16, NaHCO₃ 10) and were cooled to approximately 4° C. in the operating room following aortic cross-clamp. Failing hearts were perfused through both coronaries, while donor hearts were perfused systemically prior to removal from body. The heart was maintained at this low temperature to preserve tissue during the 15-20 minute transportation time from the operating room to the research laboratory.

Slice Preparation

After arrival, an approximately one-inch cube of left ventricular tissue or right atrial tissue was cut in cardioplegic solution. Left ventricular tissue was taken from a region close to left anterior descending artery and circumflex artery. Atrial tissue was taken from crista terminalis below sinoatrial node region. Premade 4% agarose gel cooled at 4° C. was glued on to the tissue holder of a high precision vibrating microtome (7000 smz-2, Campden Instruments Ltd. UK). The tissue block was then mounted endocardium up on the agarose. Agarose did not prevent tissue superfusion. Slices were cut tangential to the endocardium. The cutting chamber was filled with cold (4° C.) oxygenated (100% O₂) modified Tyrode's solution with excitation contraction uncoupler 2,3-butanedione (Tyrode solution, in mM: NaCl 140; KCl 6; glucose 10; HEPES 10; MgCl₂ 1; CaCl₂ 1.8; BDM 10; pH7.4). The outer chamber was constantly refilled with ice to maintain constant 4° C. in the cutting chamber.

Microtome was pre-set to 380 μm cutting thickness, 0.02-0.03 mm/s advance speed, 2 mm horizontal vibration amplitude, and 80 Hz vibration frequency. The microtome's z-axis vibration was also calibrated prior to each experiment with ceramic cutting blade to <0.5 μm. This limited cutting damage to a single layer of cardiomyocytes during cutting procedure.

After cutting, each slice was transferred immediately to oxygenated (100% O₂) washout solution at 20° C. (Tyrode solution, in mM: NaCl 140; KCl 4.5; glucose 10; HEPES 10; MgCl₂ 1; CaCl₂ 1.8; 1% Penicillin-Streptomycin; pH7.4). Slices were placed in 100 μm nylon mesh cell strainer in 6 well plates with bottom drilled through for oxygenation. Meshed washer was placed on top of the slice to prevent tissue from curling. The tissue slices were kept in the washing solution for 20 minutes to washout the BDM and warm tissue gradually to room temperature. Tissue could stay in washout solution at room temperature for up to 12 hours for acute electrophysiology studies.

Optical Mapping

Ventricular slices were electrophysiologically studied with optical mapping technique. Optical data was acquired using Ultima-L CMOS camera system (SciMedia). Transmembrane potential (Vm) was measured using di4-ANEEPS and RH237 (Life Technologies). Intracellular calcium was measured using Rhod-2AM (Life Technologies). Dye were loaded very slowly on top of the tissue and allowed to incubate for up to 30 minutes. The excitation-contraction uncoupler, blebbistatin (Cayman Chemical), was also loaded in a similar manner Using a 50 mm Nikon lens, a 1×1 cm field of view was projected to a 100×100 pixel CMOS sensor. This ensured that nearly the entire field of view was taken up by tissue to maximize resolution. Optical action potential was captured every 10 minutes during the dye incubation period to ensure optimal signal quality.

Acute Optical Mapping for Electrophysiology and Drug Response

Acute electrophysiology measurement included optical imaging of Vₘ and Ca under restitution protocol. These parameters included action potential duration (APD), activation sequence, calcium transient (CaT) duration and morphology, conduction velocity (CV), restitution properties, and effective refractory period (ERP). Basic drug response was examined using saturated dosage of isoproterenol (100 nM). All ventricular slices did not exhibit any automaticity and were paced with custom designed platinum rod field/array pacing chamber. Pacing amplitude was set at twice the amplitude of pacing threshold and 2 ms pulse width. Some atrial slices from the crista terminalis exhibited automaticity, but they were paced to acquire standardized action potential or calcium transient characteristics.

Culture Procedure and Materials

Slices were cultured in medium 199 supplemented with 2% penicillin-streptomycin, 1×ITS (Insulin, Transferrin, Selenium) liquid media supplement, and 10 mM 2,3-butanedione monoxime. Slices were individually washed in sterile PBS solution 6 times before placing in culture. Sterile forceps were used to handle slices at all time. Slices were cultured at liquid-air interface using porous transwell inserts (PICMORGO, Millipore, USA). Inserts were placed in 6-well culture plates with 1.1 ml culturing medium and placed in a 37° C. incubator with humidified air with 5% CO₂. The culture medium was changed daily.

Post Culture Electrophysiology

After a 24 hour culture period, slices' electrophysiology was measured with optical mapping to examine potential functional changes occurred during culture. APD, action potential morphology, and restitution properties were examined following the same protocol as under acute conditions.

Chronic Drug Model

Utilizing cultured slices and adult dosage of phenylephrine, a widely used selective α₁-adrenergic receptor agonist, a model to study chronic drug effects in human cardiac slices was developed. Slice electrophysiology was first acutely measured using optical mapping after application of 3 μM phenylephrine. Then acute slices were placed in either control culture with standard culture medium described above or drug medium including 3 μM phenylephrine.

Slices were cultured in a tissue incubator for 24 hours. Control cultured slices were measured before and after phenylephrine application similar to acute slices, slices cultured in drug media were measured separately. Custom made platinum tipped electrodes were made for point stimulation of slices. This allowed for more accurately characterizing conduction velocity. Only transverse conduction velocity could be measured accurately due to the small size of the tissue.

Adenoviral Transduction and Expression of Green Fluorescent Protein

To increase depth of viral tissue penetration and achieve greater virus transduction efficiency, slices were treated with collagenase after they were placed on transwell inserts. 15 μL of Type 2 collagenase (Worthington Biochem) reconstituted at 250 units/mL in slice culture medium was pipetted onto each slice and placed in a tissue incubator for 20 minutes. The slices were subsequently washed in the wells after the incubation period, followed by addition of 1.1 mL of the culture medium to the Transwell. Ad5-eGFP (total of 1.11e10 viral particles) encoding eGFP and luciferase driven by the cytomegalovirus (CMV) promoter was diluted in a total volume of 12 μL PBS, pipetted onto each slice to cover completely, and incubated on the slice for 12 hours.

Histology and Immunohistochemistry

Immunohistochemistry was performed using antibodies directed against Connexin 43 (Life Technologies 710700, 1:100 dilution) and alpha-actinin (Sigma A2172 MFCD00164521, 1:100 dilution).

Data Analysis Methods

Custom data analysis program, Rhythm, developed by our laboratory for optical mapping data was used to analyze Vₘ and Ca as described in Laughner et al. (2012. Am. J. Physiol. Heart Circ. Physiol. 303(7):H753-65). Action potential and calcium transient optical signals were low-pass filtered at 100-150 Hz, spatially averaged at 3×3 pixels, normalized from 0 to 1, and fluorescent drift was removed with a first-order fitted curve, if needed. Statistical test is performed using paired T test of unequal variance.

Activation map was plotted via calculating activation time points at 50% of action potential upstroke amplitude. Action potential and calcium transient duration was calculated at 80% repolarization/relaxation. When slices were field-paced, conduction velocity could not be quantified via traditional longitudinal and transverse direction. During point pacing conduction velocity was calculated using a semi-automated Matlab function ORCA. Effective refractory period was measured using S1-S1 pacing protocol until 1:1 capture was lost. Action potential and calcium transient were recorded at cycle lengths from 2000 ms to 200 ms.

Results

Atrial slices were taken from the crista terminalis region bordering sinus node, while ventricular slices were taken from the left or right ventricular free wall (see FIG. 4A, for example). The block of tissue was placed on a tissue holder with a back support, which prevented the tissue from sliding during slicing. Slices were cut tangentially to the epicardium while paying careful attention to the layer (i.e. subendocardial, subepicardial), because cellular electrophysiology is known to change gradually from the epicardium to the endocardium. Slices were placed in recovery solution, and after the recovery period, acute recordings were performed in different tissue layers to determine baseline electrophysiology.

Quantification of Electrophysiology Utilizing Optical Mapping

The robustness of this model for applied human research is illustrated by a broad electrophysiological assessment of ventricular slices (see e.g., FIG. 4B). Slices from the left ventricular free wall from either donor or failing hearts were sectioned. Electrophysiology was mapped with high spatial and temporal resolution either acutely, or after 24 hours of culture, with or without pharmacological treatment. Activation was measured at 50% of depolarization, and conduction velocity (CV) was measured from the activation sequence. AP duration (APD) was calculated by measuring the time between activation and repolarization completed by 80%.

To determine whether slices preserve drug responsiveness, well-characterized drugs were employed. A typical response to β-adrenergic stimulation is increased CV and APD shortening. Donor left ventricular slices paced at 1 Hz (60 beats per minute) exhibited a CV of 0.67±0.03 m/s (±S.D.) at baseline (n=13), with an increased CV of 0.84±0.03 m/s during β-adrenergic stimulation with isoproterenol (n=4) (p=0.0092, two tailed t-test assuming unequal variances). Heart failure caused slowing of CV at baseline to 0.47±0.008 m/s (n=7), but isoproterenol increased CV to 0.54±0.006 m/s (n=3, p=0.022). Culture for 24 hours did not affect CV in donor slices (0.66±0.004 m/s, n=5) versus baseline. Culture degraded the electrical properties of failing slices, therefore failing slices were not included in subsequent culture studies. These results demonstrate multiparametric optical mapping for the first time in human atrial and ventricular slices both acutely and after culture.

A typical map of slice ventricular APD demonstrated a similar baseline APD in slices (337±23 ms, n=10) when compared with the “gold standard” coronary perfused left ventricular wedge preparations (322±12 ms, n=7). β-adrenergic stimulation of slices resulted in a significant decrease of APD from a baseline to 284±20 ms (n=5) (p=0.0098), similar to the decrease from baseline to 290±16 (n=7) ms seen in wedge preparations (p=0.0082). APD was also maintained after 24 hours in culture (337±23 ms, n=10) when compared with acute slices (339±28 ms, n=5, p=0.22), which validated this model for use in chronic studies of repolarization (QT toxicity).

Multi-Parametric Optical Mapping

Multi-parametric optical mapping was tested in cardiac slices by combining transmembrane potential (Vₘ) sensitive dye and cytosolic calcium (Ca) sensitive dye. Using optical AP and calcium transient (CaT) recordings, restitution properties (waveform duration versus stimulation cycle length) revealed a significant difference in donor versus failing CaT duration (321±31 ms vs 418±34 ms; n=3, p=0.0062). APD and CaT duration were significantly different between donor and failing hearts at most pacing cycle lengths, which is similar to previous reports utilizing wedge preparations, providing further validation of the tissue slice model. β-adrenergic stimulation shifted the restitution curve downwards in both donor and failing slices. Culture did not change restitution properties of donor slices or failing slices.

Characterization of Atrial Muscle and Sino-Atrial Nodal Electrophysiology

One of the critical advantages of the cardiac slice model disclosed herein is the ability to study any region of the heart regardless of availability of a branch of coronary artery as required for perfusion in wedge preparations and/or size of the tissue sample. Tissue slices as described herein can be sustained by superfusion as their thickness is below the diffusion limit of oxygen. FIGS. 4A-4D illustrates the ability to optically map either AP or CaT in superfused atrial and SA nodal slices. A slice from the CT region bordering SA node is illustrated in FIG. 4A. In contrast to the ventricular optical AP and CaT, which are relatively homogeneous within each slice, optical CaT from the CT region consisting of both SA nodal and working atrial tissue exhibits significant heterogeneity in AP and CaT morphology and duration (FIGS. 4B and 4C). Two distinctly different upstroke durations and morphologies of CaT were observed: As discussed above, SA nodal cells exhibited slow upstrokes and long duration of CaT, while the working atrial myocardium exhibited faster upstrokes and shorter CaT duration. Furthermore, restitution pacing revealed heterogeneous effective refractory periods. At long pacing cycle length, 1:1 capture were observed in all regions. As pacing cycle length decreased, SA nodal tissue first lost 1:1 capture and then exhibited automaticity at ˜1 Hz, which corresponds to a normal heart rate. Simultaneously, the atrial muscle regions maintained 1:1 response. The inherent automaticity of slices from the SA node was also determined. FIG. 4D illustrates optical AP recordings recorded during pacing at long cycle length of 2000 ms (30 beats per minute), where both capture of paced beats as well beats originating from competing SA node firing once a second were observed. With no stimulation, spontaneously SA nodal automaticity was observed (FIG. 4D). In contrast, ventricular and atrial muscle slices lacking the SA nodal region were electrically quiescent. Restitution properties were quantified in donor atrial muscle tissue for both AP and CaT. At 1 Hz stimulation, APD was 234±27 ms (n=3) and CaT duration was 219±19 ms (n=3). These atrial durations were significantly shorter than donor left ventricular APD (p=0.0032) and CaT duration (p=0.0021), in accordance with previous published APD data from coronary perfused preparations. It is believed that these results represent the first report of CaT data from both SA node and atria of the human heart.

The results presented here using human cardiac slices show that acute versus chronic application of phenylephrine result in opposite effect on APD. Isolated human primary cells and human coronary perfused preparations have a short lifetime, precluding long-term studies involving proliferative signaling. Animal models and human iPSC-derived cardiomyocytes could be used for chronic remodeling studies, but they offer limited recapitulation of adult human physiology. Thus, organotypic human cardiac slice offers a unique clinically relevant model for both acute and chronic human testing ex vivo.

Gene therapy has been advocated for numerous diseases; however, clinical translation in the cardiovascular field is lacking. Using a GFP vector as a proof of concept approach demonstrates that an organotypic human heart slice can be used for pre-clinical validation of both cardiac efficacy and toxicity studies. A variety of candidate gene therapy strategies can be tested in this preparation prior to embarking on risky and costly human clinical trials.

Adult human atrial and ventricular slices provide a versatile and potentially high throughput model for pre-clinical trials of novel and candidate therapeutic strategies. Acute administration of β-stimulation showed expected response on the APD and CV. Unexpectedly, acute versus chronic α₁-stimulation had opposite effects on APD and CV. The cardiac tissue slice platform of the present disclosure offers a unique adult human-specific platform for studying both acute (i.e. flight-or-flight) and chronic (i.e. proliferative) signaling at the tissue level. Various putative therapies can be tested in such platform, including pharmacological therapy, virally-medicated gene therapy, cell therapy, tissue engineering, microRNA, gene editing, device therapy, among others.

This compositions and methods disclosed herein enable the foundation for tissue regeneration therapy for children and adults with SND. The compositions and methods integrate reprogramming approaches to modulate the electrophysiological properties of cardiac tissues. The compositions and methods using the gutless adenovirus enable efficient immune-system evasion and avoid toxicity. Additionally, the use of tissue-specific promoters together with the use of inducible enable the expression of transcription factors only in the presence of a well-tolerated drug will also avoid off-target effects. The compositions and methods disclosed herein further allow for the evaluation of candidate cardiac therapies in a primary substrate system. In particular, the cardiac tissue slice culture system enables the evaluation of candidate cardiac therapies as demonstrated with the adenovirus-mediated transcription factor expression systems described herein. Not only does the cardiac tissue slice culture system provide proof of concept for the compositions and methods of the present disclosure for the induction of cardiomyocytes to pacemaker-like cells, but also provides a platform for evaluating other candidate cardiac therapies such as gene therapies and drug therapies.

1.-56. (canceled)

57. A composition comprising: an adenovirus encoding a transcription factor, wherein the transcription factor comprises a β-catenin, T-box18 (Tbx18), short stature homeobox 2 (Shox2), Islet-1 (ISL1), and combinations thereof.

58. The composition of claim 57, wherein the β-catenin is a constitutively active β-catenin.

59. The composition of claim 57, wherein the adenovirus is a polycistronic adenovirus.

60. The composition of claim 57, wherein the adenovirus is a replication-defective adenovirus.

61. The composition of claim 57, wherein the adenovirus comprises a modification of the viral capsid, wherein the modification comprises an RGD sequence; polylysine residues; a serotype Ad5 knob and a serotype Ad3 knob; a binding motif that specifically binds to a cell-surface glycan; an antibody that specifically binds to a cardiomyocyte cell-surface receptor; a CD47 peptide; and combinations thereof.

62. The composition of claim 57, further comprising a carrier.

63. The composition of claim 57, further comprising a protease.

64. The composition of claim 57, further comprising a small molecule.

65. The composition of claim 57, wherein the adenovirus further encodes a tissue-specific promoter.

66. The composition of claim 57, wherein the adenovirus further encodes an inducible promoter.

67. A cardiac slice culture system comprising: a cardiac tissue slice and an incubation apparatus.

68. The cardiac slice culture system of claim 67, wherein the cardiac tissue slice is selected from the group consisting of an atrial tissue slice, a ventricle tissue slice, and combinations thereof.

69. The cardiac slice culture system of claim 67, wherein the cardiac tissue slice is positioned at a liquid-air interface.

70. The cardiac slice culture system of claim 67, wherein the cardiac tissue slice ranges from about 200 μm thick to about 400 μm thick.

71. The cardiac slice culture system of claim 67, further comprising an electrode to provide electrical stimulation to the cardiac tissue slice.

72. The cardiac slice culture system of claim 67, further comprising a recording circuit.

73. A method for evaluating a candidate cardiac therapy, the method comprising: providing a cardiac tissue slice; contacting the cardiac tissue slice with a candidate cardiac therapy; and analyzing the cardiac tissue slice.

74. The method of claim 73, wherein the cardiac tissue slice is positioned at a liquid-air interface.

75. The method of claim 73, further comprising an electrode to provide electrical stimulation to the cardiac tissue slice.

76. The method of claim 73, further comprising a recording circuit.


SEQUENCE LISTING (7 sequences)

SEQ ID NO: 1 - protein, 9 aa, Artificial Sequence
  Synthetic
CDCRGDCFC

SEQ ID NO: 2 - protein, 8 aa, Artificial Sequence
  Synthetic
GNTLSQNV

SEQ ID NO: 3 - protein, 26 aa, Artificial Sequence
  Synthetic
VYSRLNEIDTKQTTVESDISAIKTSI

SEQ ID NO: 4 - protein, 4 aa, Artificial Sequence
  Synthetic
SQNV

SEQ ID NO: 5 - protein, 21 aa, Artificial Sequence
  Synthetic
GNYTCEVTELTREGETIIELK

SEQ ID NO: 6 - DNA, 34362 bp, Artificial Sequence
  Synthetic
  misc_feature (4)..(6): n is a, c, g, or t
  misc_feature (346)..(348): n is a, c, g, or t
  misc_feature (1936)..(1938): n is a, c, g, or t
  misc_feature (2257)..(2258): n is a, c, g, or t
  misc_feature (4579)..(4581): n is a, c, g, or t
  misc_feature (34352)..(34357): n is a, c, g, or t
taannntcccttccagctctctgccccttttggattgaagccaatatgataatgagggggtggagtttgtgacgtggcgcgggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannncgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatctgagtccggacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattannncgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatctgagtccggacttgtacagctcgtccatgccgagagtgatcccggcggcggtcacgaactccanngatcggatatcttatctagaattacacggcgatctttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgcgacacctgcgtcgaagatgttggggtgttggagcaagatggattccaattcagcgggagccacctgatagcctttgtacttaatcagagacttcaggcggtcaacgatgaagaagtgttcgtcttcgtcccagtaagctatgtctccagaatgtagccatccatccttgtcaatcaaggcgttggtcgcttccggattgtttacataaccggacataatcataggacctctcacacacagttcgcctctttgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgcctgatacctggcagatggaacctcttggcaaccgcttccccgacttccttagagaggggagcgccaccagaagcaatttcgtgtaaattagataaatcgtatttgtcaatcagagtgcttttggcgaagaaggagaatagggttggcaccagcagcgcactttgaatcttgtaatcctgaaggctcctcagaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctcacgcaggcagttctatgaggcagagcgacacctttaggcagaccagtagatccagaggagttcatgatcagtgcaattgtcttgtccctatcgaaggactctggcacaaaatcgtattcattaaaaccgggaggtagatgagatgtgacgaacgtgtacatcgactgaaatccctggtaatccgttttagaatccatgataataattttttggatgattgggagctttttttgcacgttcaaaattttttgcaacccctttttggaaacgaacaccacggtaggctgcgaaatgcccatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtactcagcgtaagtgatgtccacctcgatatgtgcatctgtaaaagcaattgttccaggaaccagggcgtatctcttcatagccttatgcagttgctctccagcggttccatcttccagcggatagaatggcgccgggcctttctttatgtttttggcgtcttccatggtggctttaccaacagtaccggaatgccaagcttaggctcgagcggccgcgtcgacggtaccagatctctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccaacgcggaactccatatatgggctatgaactaatgaccccgtaattgattactattannntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatcctctagttataactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaacgatcacactaaacggtacacaggaaacaggagacacaacttgtgactgccgcggagactgtttctgcccatctgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnnnnttaat

SEQ ID NO: 7 - DNA, 37197 bp, Artificial Sequence
  Synthetic
  misc_feature (2069)..(2071): n is a, c, g, or t
  misc_feature (2390)..(2391): n is a, c, g, or t
  misc_feature (4712)..(4714): n is a, c, g, or t
taacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgagtctagcaagctagcttgatcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatctgagtccggacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattannncgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatctgagtccggacttgtacagctcgtccatgccgagagtgatcccggcggcggtcacgaactccanngatcggatatcttatctagaattacacggcgatctttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgcgacacctgcgtcgaagatgttggggtgttggagcaagatggattccaattcagcgggagccacctgatagcctttgtacttaatcagagacttcaggcggtcaacgatgaagaagtgttcgtcttcgtcccagtaagctatgtctccagaatgtagccatccatccttgtcaatcaaggcgttggtcgcttccggattgtttacataaccggacataatcataggacctctcacacacagttcgcctctttgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgcctgatacctggcagatggaacctcttggcaaccgcttccccgacttccttagagaggggagcgccaccagaagcaatttcgtgtaaattagataaatcgtatttgtcaatcagagtgcttttggcgaagaaggagaatagggttggcaccagcagcgcactttgaatcttgtaatcctgaaggctcctcagaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctcacgcaggcagttctatgaggcagagcgacacctttaggcagaccagtagatccagaggagttcatgatcagtgcaattgtcttgtccctatcgaaggactctggcacaaaatcgtattcattaaaaccgggaggtagatgagatgtgacgaacgtgtacatcgactgaaatccctggtaatccgttttagaatccatgataataattttttggatgattgggagctttttttgcacgttcaaaattttttgcaacccctttttggaaacgaacaccacggtaggctgcgaaatgcccatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtactcagcgtaagtgatgtccacctcgatatgtgcatctgtaaaagcaattgttccaggaaccagggcgtatctcttcatagccttatgcagttgctctccagcggttccatcttccagcggatagaatggcgccgggcctttctttatgtttttggcgtcttccatggtggctttaccaacagtaccggaatgccaagcttaggctcgagcggccgcgtcgacggtaccagatctctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccaacgcggaactccatatatgggctatgaactaatgaccccgtaattgattactattannntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactggggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggatggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaagctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccataccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaacttgtgactgccgcggagactgtttctgcccatctgcatactctatgtcattttcatgggactggtctggccacaactacattaatgaaatatttgccacatcctcttacactttttcatacattgcccaagaaggatcaggatcaggttcagggagtggctctaaaaagaagaaaaagaagaagtaataaagtaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaat